SEQUENCE LISTING (18 sequences)

SEQ ID NO: 1 - DNA, 20 bp, synthetic construct
  source 1..20: mol_type = other DNA
  modified_base 12: mod_base = i
  modified_base 18: mod_base = i
ccattattgtgnccctgngc

SEQ ID NO: 2 - DNA, 20 bp, synthetic construct
  source 1..20: mol_type = other DNA
  modified_base 12: mod_base = i
  modified_base 18: mod_base = i
ccattgttatgnccctgngc

SEQ ID NO: 3 - DNA, 20 bp, synthetic construct
  source 1..20: mol_type = other DNA
  modified_base 12: mod_base = i
  modified_base 18: mod_base = i
ccattattatgnccctgngc

SEQ ID NO: 4 - DNA, 20 bp, synthetic construct
  source 1..20: mol_type = other DNA
  modified_base 12: mod_base = i
  modified_base 18: mod_base = i
ccattattatgnccttgngc

SEQ ID NO: 5 - DNA, 23 bp, synthetic construct
  source 1..23: mol_type = other DNA
  modified_base 3: mod_base = i
  modified_base 9: mod_base = i
gtngtatcnacaacagtaacaaa

SEQ ID NO: 6 - DNA, 23 bp, synthetic construct
  source 1..23: mol_type = other DNA
  modified_base 3: mod_base = i
  modified_base 9: mod_base = i
gtngtatcnactacagtaacaaa

SEQ ID NO: 7 - DNA, 18 bp, synthetic construct
  source 1..18: mol_type = other DNA
sytgrgtttcyctrcgts

SEQ ID NO: 8 - DNA, 20 bp, synthetic construct
  source 1..20: mol_type = other DNA
ggttckgctygtcsrgctgg

SEQ ID NO: 9 - DNA, 17 bp, synthetic construct
  source 1..17: mol_type = other DNA
gacttctgagtctcttt

SEQ ID NO: 10 - DNA, 20 bp, synthetic construct
  source 1..20: mol_type = other DNA
gaacacctacgttctcaccg

SEQ ID NO: 11 - DNA, 21 bp, synthetic construct
  source 1..21: mol_type = other DNA
tgactgaacaatgcctcccgc

SEQ ID NO: 12 - DNA, 19 bp, synthetic construct
  source 1..19: mol_type = other DNA
cacctgatatagagtccaa

SEQ ID NO: 13 - DNA, 18 bp, synthetic construct
  source 1..18: mol_type = other DNA
cagtgccggaattatctc

SEQ ID NO: 14 - DNA, 18 bp, synthetic construct
  source 1..18: mol_type = other DNA
caccatgacccgcagacg

SEQ ID NO: 15 - DNA, 21 bp, synthetic construct
  source 1..21: mol_type = other DNA
gagaaatgagcgtggacatcc

SEQ ID NO: 16 - DNA, 21 bp, synthetic construct
  source 1..21: mol_type = other DNA
tgaaagctggcgttacggtta

SEQ ID NO: 17 - DNA, 19 bp, synthetic construct
  source 1..19: mol_type = other DNA
gtagtcctgcttgtagata

SEQ ID NO: 18 - DNA, 20 bp, synthetic construct
  source 1..20: mol_type = other DNA
tgcaacgcgcgcccttttca

(19) United States
(12) Patent Application Publication
(10) Pub. No.: US 2026/0035732 A1
(43) Pub. Date: Feb. 5, 2026

(54) METHODS, SYSTEMS, AND COMPOSITIONS FOR DETECTION OF NUCLEIC ACIDS

(71) Applicant: Reddot Bio, Inc., San Carlos, CA (US)

(72) Inventor: Caio BRUNO QUINTA DE SOUZA LEAL, Goiania, Goias (BR)

(21) Appl. No.: 19/100,326

(22) PCT Filed: Aug. 1, 2023
(86) PCT No.: PCT/US2023/071471

§ 371 Date: Jan. 31, 2025

Related U.S. Application Data
(60) Provisional application No. 63/370,218, filed on Aug. 2, 2022.

(51) Int. Cl. C12Q 1/6816; C12Q 1/689; C12Q 1/70

(52) U.S. Cl. CPC C12Q 1/6816; C12Q 1/689; C12Q 1/708; C12Q 2600/156

(57) ABSTRACT

Provided herein are methods, compositions and systems for the detection of nucleic acids. The methods compositions, and systems may comprise nanoparticles that comprise oligonucleotides. The oligonucleotides may anneal to the target nucleic acids. The nanoparticle may comprise an optical parameter that may change upon reaction with target nucleic acids.

### CROSS-REFERENCE

This application claims the benefit of priority to U.S. Provisional App. No. 63/370,218 filed Aug. 2, 2022, which is incorporated by reference herein in its entirety.

### SEQUENCE LISTING

The present application is being filed along with a Sequence Listing in electronic format. The Sequence Listing is provided as a file entitled 64978-701.601.xml, created Jul. 31, 2023, which is 21,233 bytes in size. The information in the electronic format of the Sequence Listing is incorporated by reference in its entirety.

### BACKGROUND

Nucleic acids are found in many organisms and may allow for organisms to replicate or encode for particular proteins. For example, viruses may use nucleic acids to replicate and the presence of nucleic acids of a virus in a subject may be indicative of the presence of the virus in the subject.

### SUMMARY

In an aspect the present disclosure provides a method for processing or analyzing a sample, the method comprising: (a) contacting the sample with a composition that comprises one or more nanoparticles assembled with one or more oligonucleotides to provide a test composition, wherein the one or more oligonucleotides hybridize to one or more target nucleic acid, if present, in the bodily sample; in the presence of the one or more target nucleic acids, forming a nanoparticle matrix from the one or more nanoparticles hybridized to the one or more target nucleic acids; (c) determining an optical parameter of the test composition that is indicative of the presence or absence of the one or more nucleic acids in the sample. In some embodiments, the optical parameter is determined by a color spacing analysis. In some embodiments, the optical parameter comprises absorbance, transmission, scattering, or reflection of a light at a wavelength or a range of wavelengths. In some embodiments, the optical parameter comprises a luminosity parameter (e.g., brightness of a color), a saturation parameter (e.g., intensity of a color), or a tonality parameter (e.g., shade of a color). In some embodiments, (c) further comprises comparing the optical parameter of the test composition with a corresponding optical parameter determined from a corresponding reference composition. In some embodiments, the method determines the presence or absence of the one or more target nucleic acids in the sample at a sensitivity of at least 90%, 91%, 92%, 93%, 94%, 95%, 96%, 97%, 98%, or 99%. In some embodiments, the method determines the presence or absence of the one or more target nucleic acids in the sample at a specificity of at least 90%, 91%, 92%, 93%, 94%, 95%, 96%, 97%, 98%, or 99%. In some embodiments, the method determines the presence or absence of the one or more target nucleic acids in the sample at a precision of at least 90%, 91%, 92%, 93%, 94%, 95%, 96%, 97%, 98%, or 99%. In some embodiments, the sample is selected from: a blood sample, a serum sample, a plasma sample, a saliva sample, a stool sample, a sputum sample, a urine sample, a semen sample, a transvaginal fluid sample, a cerebrospinal fluid sample, a sweat sample, a cell sample, and a tissue sample. In some embodiments, the sample is from a mammal (e.g., a human). In some embodiments, the sample is from an animal. In some embodiments, the sample is from a plant. In some embodiments, the sample comprises a lysis solution. In some embodiments, (b) comprises contacting the test composition with a nanoparticle condensation agent and/or salt. In some embodiments, the condensation agent comprises magnesium chloride. In some embodiments, at least about 40% nucleotides of the one or more oligonucleotides are guanine or cytosine. In some embodiments, about 40% to about 60% nucleotides of the one or more oligonucleotides are guanine or cytosine. In some embodiments, the one or more oligonucleotides are characterized by a melting temperature (Tm) of at least about 65 degree Celsius (° C.). In some embodiments, the one or more oligonucleotides are characterized by a Tm of about 65° C. to about 75° C. In some embodiments, the one or more oligonucleotides comprise a conjugating moiety at the 5′-end. In some embodiments, the conjugating moiety is 5′-thiol. In some embodiments, the 5′-thiol comprise a thioalkyl, such as thiohexyl. In some embodiments, a nanoparticle of the one or more nanoparticles is assembled with one, two, three, four, five, or six oligonucleotide(s). In some embodiments, the one or more nanoparticles are each (e.g., independently) assembled with one, two, three, four, five, or six oligonucleotide(s). In some embodiments, the one or more nanoparticles comprise gold. In some embodiments, the one or more nanoparticles are characterized by an average size of about 10 nanometers (nm) to about 200 nm. In some embodiments, the one or more oligonucleotides are 16 to 24 nucleotides long. In some embodiments, the one or more oligonucleotides are associated with the one or more target nucleic acid such that a distance between two adjacent nanoparticles of the one or more nanoparticles correspond to about 50 to about 70 nucleotides. In some embodiments, the one or more oligonucleotides comprises two oligonucleotides, wherein the first oligonucleotide hybridizes to a first region of the target nucleic acid, and the second oligonucleotide hybridizes to a second region of the target nucleic acid. In some embodiments, the distance between the first region and the second region of the target nucleic acid is about 50 to 70 nucleotides. In some embodiments, the one or more oligonucleotides hybridize to 10 to 30 nucleotides of the one or more target nucleic acids. In some embodiments, in the absence of one or more target nucleic acids, the one or more nanoparticles form aggregates. In some embodiments, the one or more target nucleic acids are from one or more viruses or one or more bacteria. In some embodiments, the one or more target nucleic acids are not from a coronavirus. In some embodiments, the one or more target nucleic acids are not from SARS-CoV-2 or one or more variants thereof. In some embodiments, the one or more viruses comprises an influenza virus, or a human papilloma virus. In some embodiments, the one or more bacteria comprises a Salmonella. In some embodiments, the Salmonella comprises a Salmonella enterica. In some embodiments, the Salmonella comprises one or more Salmonella strains or serovars. In some embodiments, the one or more Salmonella strains or serovars comprises one or more members selected from the group consisting of Salmonella typhimurium, Salmonella enteritidis, Salmonella gallinarum and Salmonella pullorum. In some embodiments, the one or more target nucleic acids are associated with one or more diseases or conditions. In some embodiments, the one or more diseases or conditions comprise an infectious disease, a cancer, or a degenerative disease. In some embodiments, the one or more target nucleic acids encode for a polypeptide or protein. In some embodiments, the one or more target nucleic acids comprise a DNA or RNA. In some embodiments, the DNA is a genomic DNA. In some embodiments, the RNA is a genomic RNA. In some embodiments, the RNA is a double stranded RNA or a single stranded RNA. In some embodiments, the RNA is a double stranded DNA or a single stranded DNA. In some embodiments, the one or more target nucleic acid are from human papillomavirus (HPV) or one or more variants thereof. In some embodiments, the one or more target nucleic acid comprise one or more members selected from: L1 capsid protein of HPV, L2 capsid protein of HPV, E6 protein of HPV, E7 protein of HPV, and fragments of either thereof. In some embodiments, the one or more target nucleic acids are from a Salmonella. In some embodiments, the Salmonella comprises a Salmonella enterica. In some embodiments, the Salmonella comprises one or more Salmonella strains or serovars. In some embodiments, the one or more Salmonella strains or serovars comprises one or more members selected from the group consisting of Salmonella typhimurium, Salmonella enteritidis, Salmonella gallinarum and Salmonella pullorum. In some embodiments, the one or more oligonucleotides comprise a sequence selected from SEQ ID NOS: 1-18.

In an aspect, the present disclosure provide a composition for detecting one or more target nucleic acid, the composition comprising: one or more nanoparticles assembled with one or more oligonucleotides, wherein the one or more oligonucleotides are complementary to the one or more target nucleic acid, wherein, in the presence of the one or more target nucleic acid, the one or more nanoparticles form a nanoparticle matrix, wherein the nanoparticle matrix comprises a different optical parameter compared to a solution comprising corresponding nanoparticles that are not in a nanoparticle matrix.

In another aspect, the present disclosure provides a composition for detecting a target nucleic acid, the composition comprising: one or more nanoparticles assembled with one or more oligonucleotides, wherein a first oligonucleotide of the one or more oligonucleotides is complementary to said target nucleic acid, and wherein a second oligonucleotide of the one or more oligonucleotides is complementary to said target nucleic acid at a second sequence, wherein the one or more nanoparticles comprise gold, wherein, in the presence of the one or more target nucleic acid, the one or more nanoparticles form a nanoparticle matrix. In some embodiments, at least about 40% (e.g., about 40% to about 60%)) nucleotides of the one or more oligonucleotides are guanine or cytosine. In some embodiments, the one or more oligonucleotides are characterized by a melting temperature (Tm) of at least about 65 degree Celsius (° C.) (e.g., of about 65° C. to about 75° C.). In some embodiments, the one or more oligonucleotides comprise a conjugating moiety at the 5′-end. In some embodiments, the conjugating moiety is 5′-thiol. In some embodiments, the 5′ thiol is a thioalkyl group. In some embodiments, the thioalkyl is a thiohexyl group. In some embodiments, a nanoparticle of the one or more nanoparticles is assembled with one, two, three, four, five, or six oligonucleotide(s). In some embodiments, the one or more nanoparticles are each (e.g., independently) assembled with one, two, three, four, five, or six oligonucleotide(s). In some embodiments, the one or more nanoparticles comprise gold. In some embodiments, the one or more nanoparticles are characterized by an average size of about 10 nanometers (nm) to about 200 nm. In some embodiments, the one or more oligonucleotides are 16 to 24 nucleotides long. In some embodiments, the one or more oligonucleotides are associated with the one or more target nucleic acid such that a distance between two adjacent nanoparticles of the one or more nanoparticles correspond to about 50 to about 70 nucleotides. In some embodiments, the one or more oligonucleotides comprises two oligonucleotides, wherein the first oligonucleotide hybridizes to a first region of the target nucleic acid, and the second oligonucleotide hybridizes to a second region of the target nucleic acid. In some embodiments, the distance between the first region and the second region of the target nucleic acid is about 50 to 70 nucleotides In some embodiments, one or more target nucleic acids are from one or more viruses or one or more bacteria. In some embodiments, the one or more target nucleic acids are not from a coronavirus. In some embodiments, the one or more target nucleic acids are not from SARS-CoV-2 or one or more variants thereof. In some embodiments, the one or more viruses comprises an influenza virus, or a human papilloma virus. In some embodiments, the one or more bacteria comprises a Salmonella. In some embodiments, the Salmonella comprises a Salmonella enterica. In some embodiments, the Salmonella comprises one or more Salmonella strains or serovars. In some embodiments, the one or more Salmonella strains or serovars comprises one or more members selected from the group consisting of Salmonella typhimurium, Salmonella enteritidis, Salmonella gallinarum and Salmonella pullorum. In some embodiments, the one or more target nucleic acids are associated with one or more diseases or conditions. In some embodiments, the one or more diseases or conditions comprise an infectious disease, a cancer, or a degenerative disease. In some embodiments, the one or more target nucleic acids encode for a polypeptide or protein. In some embodiments, the one or more target nucleic acids comprise a DNA or RNA. In some embodiments, the DNA is a genomic DNA. In some embodiments, the RNA is a genomic RNA. In some embodiments, the RNA is a double stranded RNA or a single stranded RNA. In some embodiments, the RNA is a double stranded DNA or a single stranded DNA. In some embodiments, the one or more target nucleic acid are from human papillomavirus (HPV) or one or more variants thereof. In some embodiments, the one or more target nucleic acid comprise one or more members selected from: L1 capsid protein of HPV, L2 capsid protein of HPV, E6 protein of HPV, E7 protein of HPV, and fragments of either thereof. In some embodiments, the one or more target nucleic acids are from a Salmonella. In some embodiments, the Salmonella comprises a Salmonella enterica. In some embodiments, the Salmonella comprises one or more Salmonella strains or serovars. In some embodiments, the one or more Salmonella strains or serovars comprises one or more members selected from the group consisting of Salmonella typhimurium, Salmonella enteritidis, Salmonella gallinarum and Salmonella pullorum. In some embodiments, the one or more oligonucleotides comprise a sequence selected from SEQ ID NOS: 1-18.

In another aspect, the present disclosure provides a kit for identifying the presence of a target nucleic acid, the kit comprising: (i) one or more gold nanoparticles assembled with one or more oligonucleotides, (ii) a condensation solution, (iii) instructions for using said one or more gold nanoparticles assembled with one or more oligonucleotides.

In another aspect, the present disclosure provides a kit for identifying the presence of a target nucleic acid, the kit comprising: (i) the compositions described elsewhere herein, (ii) a condensation solution, (iii) instructions for using said one or more gold nanoparticles assembled with one or more oligonucleotides.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “figure” and “FIG.” herein), of which:

FIGS. 1A-1B show an example schematic of the methods disclosed herein.

FIG. 2A shows a representation of a negative sample. FIG. 2B shows a representation of a positive sample. FIG. 2C shows a series of cuvettes with increasing amount of positive signal.

FIG. 3 shows example nanoparticle configurations.

FIG. 4 shows UV-visible spectra of gold nanoparticles coupled to oligonucleotides.

FIG. 5 shows an example device for performing the methods of the disclosure.

FIG. 6 shows the results of an assay detecting nucleic acid sequences of HPV.

FIG. 7A-7C shows data of an assay for detection of HPV using optical density. FIG. 7A shows averages measurements of the samples, FIG. 7B shows a curve for each sample in the CasKi assay, FIG. 7C shows a curve for each sample in the HeLa assay.

FIG. 8A-8B shows data of an assay for detection of HPV using optical density. FIG. 8A shows an ROC curve for the assay using CasKi cells, FIG. 8B shows an ROC curve for the assay using HeLa cells.

FIG. 9 shows data of an assay for detection of Salmonella using optical density.

FIG. 10 shows data of an assay for detection of Salmonella using optical density.

FIG. 11 shows an ROC curve for the assay for detection of Salmonella using optical density.

FIG. 12 shows a computer control system that is programmed or otherwise configured to implement methods provided herein.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

The detection of nucleic acids has broad applications for human health. Specifically, nucleic acids are present in subjects and may be relevant to disease conditions or pathogenic infections. Nucleic acids are present in organisms or microbes for replication, as well as protein expression. Detection of a specific nucleic acid may indicate the presence of a particular microbe in a sample/subject and may be used to diagnose a subject with a condition. Detection of a human nucleic acid may also be used to identify mutations in a subject's genome and may be indicative of a disease, disorder, or other state, such as cancer. Detection of particular nucleic acids may involve complex reactions that may require specific conditions and may still provide inaccurate results.

The present disclosure provides methods, systems and compositions that allow for detection of particular nucleic acids that are rapid, easy to use, and provide accurate results. The method may provide an alternative to other nucleic acid detection techniques, and may be performed without a thermocycler or other instrument that heat and/or cool the nucleic acids. The method may also be performed without the use of enzymes allowing kits or compositions to be more easily stored and maintain a longer shelf life. The method may be performed without nucleic acid extension or amplification. As such, the methods disclosed may comprise simpler and more robust sample preparation compared to methods that use nucleic acid amplification, and may be less susceptible to reagent degradation. The methods and compositions may be used without a need for complex or expensive optical instruments that would otherwise be generally unavailable to the public or cost-prohibitively difficult to procure for a member of the general public.

The methods and compositions may allow for the rapid testing for the presence of nucleic acids compared to other methods of detection, and may for example, generate results within 5 minutes, or less. Additionally, the manufacturing costs of the compositions may be lower than other tests of similar accuracy and may be cheaper for the test to be performed. The methods may also have other advantages over methods of similar accuracy. For example, the methods may remove the need for purification of nucleic acids prior to assaying, reducing the time needed for a result to be generated. Additionally, the method may be performed without microbial culturing or storage of RNA samples.

Provided herein are methods, systems and composition for detection of nucleic acids analytes. In aspect, the present disclosure provides compositions for detecting one or more target nucleic acid (DNA or RNA), the composition comprising: one or more nanoparticles assembled with one or more oligonucleotides, wherein the one or more oligonucleotides are configured to bind the one or more target nucleic acid, wherein, in the presence of the one or more target nucleic acid, the one or more nanoparticles form a nanoparticle matrix. The nanoparticle matrix may be detected via the optical properties or parameters of nanoparticle matrix. The one or more nanoparticle in solution may have different optical properties than the nanoparticle matrix such that the formation of a nanoparticle matrix from one or more nanoparticles may be identified via the optical properties or parameters. The one or more oligonucleotides may each comprise about 16 to about 24 nucleotides. The one or more oligonucleotides may be associated with the one or more target nucleic acid such that a (e.g., average) distance between two adjacent nanoparticles of the one or more nanoparticles correspond to about 50 to about 70 nucleotides. In some cases, the one or more oligonucleotides comprises two oligonucleotides, wherein the first oligonucleotide hybridizes to a first region of the target nucleic acid, and the second oligonucleotide hybridizes to a second region of the target nucleic acid. In some embodiments, the distance between the first region and the second region of the target nucleic acid is about 50 to 70 nucleotides.

Provided herein is a method for processing or analyzing a sample of a subject, the method comprising: (a) contacting the bodily sample with a composition that comprises one or more nanoparticles assembled with one or more oligonucleotides to provide a test composition, wherein the one or more oligonucleotides; and (2) are configured to bind one or more target nucleic acid, if present, in the sample; (b) subjecting the test composition of (a) to conditions sufficient to induce aggregation of the one or more nanoparticles in the absence of the one or more target nucleic acid, wherein, in the presence of the one or more target nucleic acids, the one or more nanoparticles form a nanoparticle matrix; (c) determining an optical parameter of the test composition that is indicative of the presence or absence of the one or more target nucleic acids in the sample. The one or more oligonucleotides may each comprise about 16 to about 24 nucleotides. The one or more oligonucleotides may be associated with the one or more target nucleic acid such that a (e.g., average) distance between two adjacent nanoparticles of the one or more nanoparticles correspond to about 50 to about 70 nucleotides.

In various aspects disclosed herein, the methods may be used to determine that a nucleic acid sequence or infectious pathogen is present in a subject or sample. The methods may be used to determine the presence of an infectious pathogen in a subject or diagnose the presence of a disorder or disease.

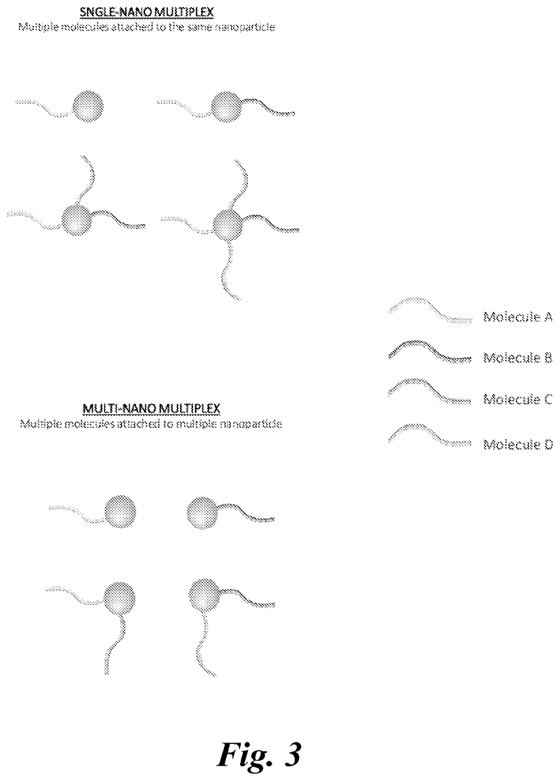

In various aspects, one or more oligonucleotides are assembled with a nanoparticle. The one or more oligonucleotides may be complementary to particular sequences. The one or more nucleotides may anneal to target sequences. In some embodiments, the annealing comprises generating a duplex between two strands of nucleic acids that form hydrogen bonds between complementary bases. After the one or more oligonucleotides anneal to a target, the resulting product may be detected in order to determine the presence of the target sequences. The oligonucleotides may be designed to bind, anneal, or hybridize to specific sequences. For example, the one or more oligonucleotides may anneal to a viral sequence. The one or more oligonucleotides may anneal to a bacterial sequence. The one or more oligonucleotides may anneal to a fungal sequence. The one or more oligonucleotides may anneal to a human sequence. These oligonucleotide sequences may be generated by analyzing target sequences and generating partially or fully complementary sequences to the target nucleic acid. The one or more oligonucleotides assembled a given nanoparticle may comprise a same sequence or a different sequence. A solution of nanoparticle may also comprise nanoparticles with the same oligonucleotides or different oligonucleotides. FIG. 3 illustrates example nanoparticles that may be used in the methods, compositions and systems of this disclosure. The single nano multiplex shows nanoparticles with different oligonucleotides attached to one nanoparticle. The multi-nano multiplex shows multiple oligonucleotide molecules attached to multiple nanoparticles.

The one or more oligonucleotides may comprise specific or particular characteristics. The one or more oligonucleotides may comprise a melting temperature. The melting temperature may be a temperature such that the one or more oligonucleotides remain annealed to the target nucleic acids at a particular assaying temperature or reaction temperature. The melting temperature may be a temperature such that the one or more oligonucleotides do not remain annealed to the target nucleic acids at a particular assaying temperature or reaction temperature. The one or more oligonucleotides may have a melting temperature (Tm) of at least about 65 degree Celsius (° C.). The one or more oligonucleotides may have a Tm of about 65° C. to about 75° C. The one or more oligonucleotides may have a Tm of no more than 65° C., 66° C., 67° C., 68° C., 69° C., 70° C., 71° C., 72° C., 73° C., 74° C., 75° C., 76° C., 77° C., 78° C., 79° C., or 80° C., or less. The one or more oligonucleotides may have a Tm of greater than 65° C., 66° C., 67° C., 68° C., 69° C., 70° C., 71° C., 72° C., 73° C., 74° C., 75° C., 76° C., 77° C., 78° C., 79° C., or 80° C., or more. The Tm of the one or more nucleotides may be related to the guanine or cytosine (GC) content of the oligonucleotides. The one or more oligonucleotides may comprise a particular percentage of a given nucleotide. For example, an oligonucleotide of the one or more oligonucleotides may comprises at least 20% guanine. An oligonucleotide of the one or more oligonucleotides may comprises at least 30% guanine. At least 40% of the nucleotides of a oligonucleotide may be guanine or cytosine. At least 45% of the nucleotides of a oligonucleotide may be guanine or cytosine. At least 50% of the nucleotides of a oligonucleotide may be guanine or cytosine. At least 55% of the nucleotides of a oligonucleotide may be guanine or cytosine. At least 60% of the nucleotides of a oligonucleotide may be guanine or cytosine. About 40% to about 60% of the nucleotides of the one or more oligonucleotides may be guanine or cytosine. The one or more oligonucleotides may comprise minimal secondary structure. For example, the one or more oligonucleotides may not have a harpin, or self-anneal. The one or more oligonucleotides may comprise minimal interactions between one another and may be designed such that oligonucleotides of the one or more oligonucleotides do not anneal to one another.

The one or more oligonucleotides may comprise or more reactive groups or conjugating moieties. The reactive groups or conjugating moieties may allow the oligonucleotide to be conjugated, attached, or otherwise assembled to another molecule. The oligonucleotides may comprise a conjugation moiety that allows it to be conjugated to a nanoparticle. The one or more oligonucleotides may comprise a conjugating moiety at the 5′-end. The one or more oligonucleotides may comprise a conjugating moiety at the 3′-end. The conjugating moiety may be thiol, for example a 3′ thiol, or a 5′-thiol, such as a thioalkyl, (e.g., thiohexyl).

The one or more oligonucleotides may comprise a particular or specific sequence. The one or more oligonucleotides may comprise a sequence of Table 1. The oligonucleotides may comprise a sequence identical or complementary to a sequence selected from SEQ ID NO: 1-18. The oligonucleotides may comprise a sequence at least 90%, at least 91%, at least 92%, at least 93%, at least 94%, at least 95%, at least 96%, at least 97%, at least 98%, or at least 99% identical or complementary to a sequence selected from SEQ ID NO: 1-18. The oligonucleotides may comprise a sequence at least 90% identical or complementary to a sequence selected from SEQ ID NO: 1-18. The oligonucleotides may comprise a sequence at least 91% identical or complementary to a sequence selected from SEQ ID NO: 1-18. The oligonucleotides may comprise a sequence at least 92% identical or complementary to a sequence selected from SEQ ID NO: 1-18. The oligonucleotides may comprise a sequence at least 93% identical or complementary to a sequence selected from SEQ ID NO: 1-18. The oligonucleotides may comprise a sequence at least 94% identical or complementary to a sequence selected from SEQ ID NO: 1-18. The oligonucleotides may comprise a sequence at least 95% identical or complementary to a sequence selected from SEQ ID NO: 1-18. The oligonucleotides may comprise a sequence at least 96% identical or complementary to a sequence selected from SEQ ID NO: 1-18. The oligonucleotides may comprise a sequence at least 97% identical or complementary to a sequence selected from SEQ ID NO: 1-18. The oligonucleotides may comprise a sequence at least 98% identical or complementary to a sequence selected from SEQ ID NO: 1-18. The oligonucleotides may comprise a sequence at least 99% identical or complementary to a sequence selected from SEQ ID NO: 1-18. The oligonucleotides may comprise no more than three, no more than two, or no more than one alteration(s) relative to a sequence selected from SEQ ID NO: 1-18 or a complementary sequence thereof. The oligonucleotides may comprise no more than three alterations relative to a sequence selected from SEQ ID NO: 1-18 or a complementary sequence thereof. The oligonucleotides may comprise no more than two alterations relative to a sequence selected from SEQ ID NO: 1-18 or a complementary sequence thereof. The oligonucleotides may comprise no more than one alteration relative to a sequence selected from SEQ ID NO: 1-18 or a complementary sequence thereof. The no more than three, no more than two, or no more than one alteration(s) may comprise substitution(s), addition(s), deletion(s), or a combination thereof. The no more than three, no more than two, or no more than one alteration(s) may be substitution(s). The one or more oligonucleotides may comprise a degenerate base or a modified base. A degenerate base may be a first base on a first oligonucleotide and a second base on a second oligonucleotide. For example, a sequence may comprise a degenerate base at a position indicated by the letter “K”. In a first oligonucleotide, a guanine may be present at the position indicated by the letter “K”, and in a second oligonucleotide, a threonine may be present at the position indicated by the letter “K”. The sequence may therefore represent a mixture of the first a second oligonucleotides. The use of a degenerate base in a sequence of an oligonucleotide may allow the one or more nucleotide to bind to a wider variety of sequences, such as a variant sequence.

TABLE 1

| SEQ | | | |
|---|---|---|---|
| ID NO: | Name | Sequence (5'-3') | Target |
| 1 | APT22DxL-1 | CCATTATTGTGiCCCTGiGC | HPV-L1 |
| 2 | APT22DxL-2 | CCATTGTTATGiCCCTGiGC | HPV-L1 |
| 3 | APT22DxL-3 | CCATTATTATGiCCCTGiGC | HPV-L1 |
| 4 | APT22DxL-4 | CCATTATTATGiCCTTGiGC | HPV-L1 |
| 5 | APT22DxL-5 | GTiGTATCiACAACAGTAACAAA | HPV-L1 |
| 6 | APT22DxL-6 | GTiGTATCiACTACAGTAACAAA | HPV-L1 |
| 7 | APT22DxE-1 | SYTGRGTTTCYCTRCGTS | HPV-E6 |
| 8 | APT22DxE-2 | GGTTCKGCTYGTCSRGCTGG | HPV-E7 |
| 9 | MDx22DS-1 | GACTTCTGAGTCTCTTT | Salmonella spp |
| 10 | MDx22DS-2 | GAACACCTACGTTCTCACCG | Salmonella spp |
| 11 | MDx23DS-ST1 | TGACTGAACAATGCCTCCCGC | Salmonella |
| | | | typhimurium |
| 12 | MDx23DS-ST2 | CACCTGATATAGAGTCCAA | Salmonella |
| | | | typhimurium |
| 13 | MDx23DS-SE1 | CAGTGCCGGAATTATCTC | Salmonella |
| | | | enteritidis |
| 14 | MDx23DS-SE2 | CACCATGACCCGCAGACG | Salmonella |
| | | | enteritidis |
| 15 | MDx23DS- | GAGAAATGAGCGTGGACATCC | Salmonella |
| | SG/P1 | | gallinarum; |
| | | | Salmonella |
| | | | pullorum |
| 16 | MDx23DS- | TGAAAGCTGGCGTTACGGTTA | Salmonella |
| | SG/P2 | | gallinarum; |
| | | | Salmonella |
| | | | pullorum |
| 17 | MDx23DS-P1 | GTAGTCCTGCTTGTAGATA | Salmonella spp |
| 18 | MDx23DS-P2 | TGCAACGCGCGCCCTTTTCA | Salmonella spp |

Degenerate Base: K = G + T R = A + G S = C + G Y = C + T; indicates a mixture of oligonucleotides, wherein for a given oligonucleotide, the position of the degenerate base is one of the possible bases listed.
Internal Modifications: C6-Spacer C6 i = Inosine

In various embodiments, the one or more oligonucleotides are configured to bind to one or more target nucleic acids. Binding to one more target nucleic acids may comprise annealing (or hybridizing) to the target nucleic acids, under conditions such to generate a double stranded nucleic acid complex. For example, one or more oligonucleotides may be configured to bind to one or more targets based at least on complementarity of the oligonucleotides to the target nucleic acids. In some embodiments, the one or more oligonucleotides hybridizes to a target nucleic acid if the oligonucleotide comprises at least about 80%, 81%, 82%, 83%, 84%, 85%, 86%, 87%, 88%, 89%, 90%, 91%, 92%, 93%, 94%, 95%, 96%, 97%, 98%, 99%, or 100% complementarity to the target nucleic acid.

The one or more oligonucleotides may be able to anneal to nucleic acids associated with a disease, such as cancer. The nucleic acid targets may be associated with a degenerative disease. The nucleic acid target may be a nucleic acid from, or derived from, an infectious agent. For example, the one or more oligonucleotides may be able to anneal to nucleic acids that comprise a sequence from, or derived from, human papilloma virus (HPV) gene. The one or more oligonucleotides may be able to anneal to nucleic acids that comprise a sequence that indicated the presence of HPV in a subject. The one or more oligonucleotides may be able to anneal to nucleic acids that comprise a sequence from a L1 capsid gene of HPV, L2 capsid gene of HPV, E6 gene of HPV, E7 gene of HPV, and fragments thereof. The one or more oligonucleotides may be able to anneal to nucleic acids that encode for proteins or polypeptides. For example, the nucleic acid that encode for protein or polypeptides may encode for the L1 capsid protein of HPV, L2 capsid protein of HPV, E6 protein of HPV, E7 protein of HPV, or fragments thereof. The one or more oligonucleotides may be able to anneal to nucleic acids that comprise a sequence that indicated the presence of Salmonella in a subject. The one or more oligonucleotides may be able to anneal to nucleic acids that comprise a sequence from, or derived from, a Salmonella species. The one or more oligonucleotides may be able to anneal to nucleic acids that comprise a sequence from, or derived from, Salmonella enterica. The one or more oligonucleotides may be able to anneal to nucleic acids that comprise a sequence from, or derived from, different species or serovars of Salmonella. For example, The one or more oligonucleotides may be able to anneal to nucleic acids that comprise a sequence from, or derived from, Salmonella typhimurium, Salmonella enteritidis, Salmonella gallinarum or Salmonella pullorum. The one or more oligonucleotides may be able to anneal to nucleic acids that comprise a sequence that is shared between two or more different species or serovars of Salmonella. For example, The one or more oligonucleotides may be able to anneal to nucleic acids that comprise a sequence shared by Salmonella gallinarum and Salmonella pullorum. In another example, the nucleic acid may comprise a sequence shared between multiples members of the Salmonella genus or multiple strains of Salmonella enterica. For example, the one or more oligonucleotides may bind to that is present is multiple strains of Salmonella enterica and therefore indicate the presence of at least on strain of Salmonella.

Combinations of the one or more oligonucleotides may be used to detect a particular target. For example, in a given mixture, multiple oligonucleotides with different sequences may be present. In some cases, the one or more oligonucleotides comprise a first oligonucleotide comprising a sequence identical or at least 90%, at least 91%, at least 92%, at least 93%, at least 94%, at least 95%, at least 96%, at least 97%, at least 98%, or at least 99% identical or complementary to a sequence of SEQ ID NO: 9, and a second oligonucleotide comprising a sequence identical or at least 90%, at least 91%, at least 92%, at least 93%, at least 94%, at least 95%, at least 96%, at least 97%, at least 98%, or at least 99% identical or complementary to a sequence of SEQ ID NO: 10. In some cases, the one or more oligonucleotides comprise a first oligonucleotide comprising a sequence identical to SEQ ID NO: 9, and a second oligonucleotide comprising a sequence identical to SEQ ID NO: 10. In some cases, the one or more oligonucleotides comprise a first oligonucleotide comprising a sequence identical or at least 90%, at least 91%, at least 92%, at least 93%, at least 94%, at least 95%, at least 96%, at least 97%, at least 98%, or at least 99% identical or complementary to a sequence of SEQ ID NO: 11, and a second oligonucleotide comprising a sequence identical or at least 90%, at least 91%, at least 92%, at least 93%, at least 94%, at least 95%, at least 96%, at least 97%, at least 98%, or at least 99% identical or complementary to a sequence of SEQ ID NO: 12. In some cases, the one or more oligonucleotides comprise a first oligonucleotide comprising a sequence identical to SEQ ID NO: 11, and a second oligonucleotide comprising a sequence identical to SEQ ID NO: 12. In some cases, the one or more oligonucleotides comprise a first oligonucleotide comprising a sequence identical or at least 90%, at least 91%, at least 92%, at least 93%, at least 94%, at least 95%, at least 96%, at least 97%, at least 98%, or at least 99% identical or complementary to a sequence of SEQ ID NO: 13, and a second oligonucleotide comprising a sequence identical or at least 90%, at least 91%, at least 92%, at least 93%, at least 94%, at least 95%, at least 96%, at least 97%, at least 98%, or at least 99% identical or complementary to a sequence of SEQ ID NO: 14. In some cases, the one or more oligonucleotides comprise a first oligonucleotide comprising a sequence identical to SEQ ID NO: 13, and a second oligonucleotide comprising a sequence identical to SEQ ID NO: 14. In some cases, the one or more oligonucleotides comprise a first oligonucleotide comprising a sequence identical or at least 90%, at least 91%, at least 92%, at least 93%, at least 94%, at least 95%, at least 96%, at least 97%, at least 98%, or at least 99% identical or complementary to a sequence of SEQ ID NO: 15, and a second oligonucleotide comprising a sequence identical or at least 90%, at least 91%, at least 92%, at least 93%, at least 94%, at least 95%, at least 96%, at least 97%, at least 98%, or at least 99% identical or complementary to a sequence of SEQ ID NO: 16. In some cases, the one or more oligonucleotides comprise a first oligonucleotide comprising a sequence identical to SEQ ID NO: 15, and a second oligonucleotide comprising a sequence identical to SEQ ID NO: 16. In some cases, the one or more oligonucleotides comprise a first oligonucleotide comprising a sequence identical or at least 90%, at least 91%, at least 92%, at least 93%, at least 94%, at least 95%, at least 96%, at least 97%, at least 98%, or at least 99% identical or complementary to a sequence of SEQ ID NO: 17, and a second oligonucleotide comprising a sequence identical or at least 90%, at least 91%, at least 92%, at least 93%, at least 94%, at least 95%, at least 96%, at least 97%, at least 98%, or at least 99% identical or complementary to a sequence of SEQ ID NO: 18. In some cases, the one or more oligonucleotides comprise a first oligonucleotide comprising a sequence identical to SEQ ID NO: 17, and a second oligonucleotide comprising a sequence identical to SEQ ID NO: 18. The combination of two or more oligonucleotides may allow multiple oligonucleotides to bind to a target nucleic acid. By binding multiple oligonucleotides to a same nucleic acid target, a structure may be formed that comprises multiple nanoparticles and may allow for the generation of a matrix or colloidal solution. As the one or more oligonucleotides can be bound to a nanoparticle, multiple nanoparticles can be bound to a single target, and similarly multiple targets can be bound to a single nanoparticle that has multiple conjugated oligonucleotides. The binding events can generate a matrix generated by nanoparticles, the target nucleic acids, and the one or more oligonucleotides. This matrix may have optical properties that are distinct from a solution without this matrix and thus the formation of a matrix may be detectable by observing the optical properties of a solution.

The optical properties of the matrix may be modulated or may be dependent upon the distance between adjacent nanoparticles. The distance between adjacent nanoparticles may be modulated via the length of the oligonucleotides. The distance may be modulated by the number of nucleotides that separate the target sequences on a given nucleic acid target. For example, as described herein, multiple oligonucleotides may bind to a nucleic acid target. A first oligonucleotide may bind to a first sequence that is multiple nucleotides away from the binding location a second oligonucleotide. A first oligonucleotide may bind a sequence that is at least 30 nucleotides away from the sequence that a second oligonucleotide binds to. A first oligonucleotide may bind a sequence that is no more than 30 nucleotides away from the sequence that a second oligonucleotide binds to. A first oligonucleotide may bind a sequence that is at least 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71,72, 73, 74, 75, 76, 77, 78, 79, 80, 81, 82, 83, 84, 85, 86, 87, 88, 89, 90, or more nucleotides away from the sequence that a second oligonucleotide binds to. A first oligonucleotide may bind a sequence that is no more than 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71,72, 73, 74, 75, 76, 77, 78, 79, 80, 81, 82, 83, 84, 85, 86, 87, 88, 89, 90, or more nucleotides away from the sequence that a second oligonucleotide binds to. A first oligonucleotide may bind a sequence that is 50 to 70 nucleotides away from the sequence that a second oligonucleotide binds to. The length of the oligonucleotides may alter the ability for a matrix to be formed based on the physical properties of the nanoparticles and oligonucleotides. For example, too short of a oligonucleotide, insufficient spacing between adjacent binding locations, may cause adjacent nanoparticles to directly contact or sterically inhibit the formation of a matrix. Similarly, the size of a nanoparticle may be relevant for the distance separating to nanoparticles. For example, smaller nanoparticles may allow for smaller distances between adjacent nanoparticles, while still generating a matrix upon binding of the target nucleic acids. As such, generation of a matrix upon binding of a target nucleic acid may be sufficient to detect the presence of the target nucleic acids, regardless of specific oligonucleotide length and nucleotide separation distances.

The one or more oligonucleotides may comprise a length or number of nucleotides. For example, the one or more oligonucleotides may be at least 10, 11, 12, 13, 14,15, 16, 17, 18, 19, 20, 21, 22, 23, 24, 25, 26, 27, 28, 29, 30, 31, 32, 33, 34, 35,36, 37, 38, 39, 40, 41, 42, 43, 44, 45, 46, 47, 48, 49, 50 or more nucleotides long. For example, the one or more oligonucleotides may be no more than 10, 11, 12, 13, 14, 15, 16, 17, 18, 19, 20, 21, 22, 23, 24, 25, 26, 27, 28, 29, 30, 31, 32, 33, 34, 35,36, 37, 38, 39, 40, 41, 42, 43, 44, 45, 46, 47, 48, 49, 50 or less nucleotides long. The oligonucleotides may be about 16 to about 24 nucleotides long. The oligonucleotides may be about 10 to about 20 nucleotides long. The oligonucleotides may be about 20 to about 30 nucleotides long.

The systems, methods, and compositions of the disclosure may comprise nanoparticles. The nanoparticles may be used to detect the presence of a target nucleic acid. The nanoparticle may comprise or otherwise be assembled with one or more oligonucleotides. Assembled may mean the nanoparticle and the oligonucleotide are conjugated to each other. Assembled may mean the nanoparticle and oligonucleotide are connected to each other. In some embodiments, a nanoparticle of the one or more nanoparticles is assembled with one, two, three, four, five, or six oligonucleotides. In some embodiments, nanoparticle of the one or more nanoparticles is assembled with at least one, two, three, four, five, or six, or more oligonucleotides. The one or more nanoparticles may each (e.g., independently) be assembled with one, two, three, four, five, or six oligonucleotide(s). For example, a first nanoparticle can comprise a first oligonucleotides and a second oligonucleotide, wherein the first and second oligonucleotides comprises different sequences. For example, a first nanoparticle can comprise a first oligonucleotides and a second nanoparticle can comprise a second oligonucleotide, wherein the first and second oligonucleotides comprises different sequences. In another example, a first nanoparticle can comprise a first oligonucleotides and a second nanoparticle can comprise a second oligonucleotide, wherein the first and second oligonucleotides comprise the same sequences.

The one or more nanoparticles may comprise a variety of materials. The nanoparticle may comprise gold. The nanoparticle may comprise a metal. The metal may have an optical property based on coordination chemistry. The metal may have an optical property based on the reflectance, absorption, or transmission of particular wavelengths. The nanoparticle may comprise a material that comprises an optical property, for example, a reflectance, transmittance. The optical property of a nanoparticle may allow for the particle to be detected in a solution. The optical property may be altered in the presence of a target nucleic acid. For example, in the presence of a target nucleic acid, the nanoparticles may create a matrix of nanoparticles that has a different optical property than a single nanoparticle.

The various compositions may comprise the use of gold nanoparticles. Given its unique stability as a pure metal, gold is practical element for use in nanoscience. Gold nanoparticles are appreciated for optical and electronic properties such as those described at https://www.sigmaaldrich.com/BR/pt/technical-documents/technical-article/materials-science-and-engineering/biosensors-and-imaging/gold-nanoparticles, Gold has exceptional optical properties, such as high extinction coefficient, chemical stability, water-solubility, localized surface plasmon resonance, and inherent photostability. The interaction or rupture between the chemical bonds of the nanometric particles with the oligonucleotides allows the revelation of positive or negative results. The observation evidenced by the departure from its original color means a negative result. The strength of binding between the conjugate and the investigated nucleic acid may determine the sensitivity and specificity of the method.

The reddish impression, characteristic of gold nanoparticles, is related to their reduced size and a large number of electrons on their surface. According to the theory, the luminous display produced by the incidence of light on the surface of conduction band nanoparticles (plasmonic resonance) that propagates with waves associated with the characteristic reception value. A surface resonance band in the visible region that arises from the oscillation of electrons in the region relative to the lattice of metal ions may be observed. Therefore, in the presence of agglomeration of particles or when the diameter increases, a color change of the solution from red to blue can be observed.

The optical and electronic properties of gold nanoparticles may be adjusted by changing the size, shape, surface chemistry, or state of aggregation. Optical interaction and development may be determined by their size and dimension. A beam of light propagating close to a colloidal nanoparticle may interact with free electrons inducing an oscillation of the electronic charge signal. This phenomenon is directly related to the frequency of visible light. Short gold nanoparticles (about 30 nm) have absorption wavelength in the blue-green portion (450 nm spectrum), while the red wavelength in the spectrum is reflected at 700 nm. When red is absorbed, blue is reflected, resulting in a blue or purple-colored solution. As the particle size increases towards the mass limit, the surface plasmon resonance wavelengths move, and the more visible wavelengths are reflected, giving the nanoparticles a light or translucent color. Surface plasmon resonance is flexible and changes depending on its application, size, or shape. The chemical interaction of gold nanoparticles in contact with saline solutions or excess salt brings neutrality to the reaction, causing aggregation of the nanoparticles. This result changes its original color, red, to blue. This problem can be corrected by desalination or protected by coating polymers, small molecules, and specific biological recognition molecules.

Gold nanoparticles (AuNPs) may comprise surface citrate molecules in solution. These surface citrate molecules may be replaced with differentially thiol-functionalized oligonucleotides, such as anti-sense oligonucleotides (ASOs). The intrinsic optical properties of AuNPs associated with targeting ability of oligonucleotides may be used to develop a selective detection platform. The reactivity of the oligonucleotides to a target may be associated with a surface resonance in the visible region and without the need for any expensive instrumental techniques. The optical change can be observed via the naked eye or using cameras or other image analysis technique as opposed to requiring the observation of specific wavelength with a more expensive instrument. Moreover the ratio of oligonucleotides (e.g., ASOs to AuNPs (ASO/AuNPs) may be modulated to tune the sensitivity of the biosensor to the target. The increase in AuNP-ASO sensitivity can be monitored using defined concentrations of target analyte (DNA/RNA) by changing the incubation temperature, knowing that the optimal sensitivity is achieved at 37° C. Observing the relative sensitivity of the nanoparticles of gold coated with ASOs, through the monitoring with the comparative increase of the absorbance at 660 nm. This makes it possible to evaluate different colloidal distribution profiles in the analyzed range.

The one or more nanoparticles may be characterized by a size. The one or more nanoparticles may be characterized by average size of about 10 nanometers (nm). The one or more nanoparticles may be characterized by average size of about 20 nm, 30 nm, 40 nm, 50 nm, 60 nm, 70 nm, 80 nm, 90 nm, 100 nm, 110, 120 nm, 130 nm, 140 nm, 150 nm, 160 nm, 170 nm, 180 nm, 190 nm, or 200 nm. The one or more nanoparticles may be characterized by average size of at least 20 nm, 30 nm, 40 nm, 50 nm, 60 nm, 70 nm, 80 nm, 90 nm, 100 nm, 110, 120 nm, 130 nm, 140 nm, 150 nm, 160 nm, 170 nm, 180 nm, 190 nm, or 200 nm, or more. The one or more nanoparticles may be characterized by average size of no more than 20 nm, 30 nm, 40 nm, 50 nm, 60 nm, 70 nm, 80 nm, 90 nm, 100 nm, 110, 120 nm, 130 nm, 140 nm, 150 nm, 160 nm, 170 nm, 180 nm, 190 nm, or 200 nm, or less. The one or more nanoparticles may be characterized by a size of about 20 nm, 30 nm, 40 nm, 50 nm, 60 nm, 70 nm, 80 nm, 90 nm, 100 nm, 110, 120 nm, 130 nm, 140 nm, 150 nm, 160 nm, 170 nm, 180 nm, 190 nm, or 200 nm. The one or more nanoparticles may be characterized by a size of at least about 10 nm 20 nm, 30 nm, 40 nm, 50 nm, 60 nm, 70 nm, 80 nm, 90 nm, 100 nm, 110, 120 nm, 130 nm, 140 nm, 150 nm, 160 nm, 170 nm, 180 nm, 190 nm, or 200 nm, or more. The one or more nanoparticles may be characterized by a size of no more than about 10 nm 20 nm, 30 nm, 40 nm, 50 nm, 60 nm, 70 nm, 80 nm, 90 nm, 100 nm, 110, 120 nm, 130 nm, 140 nm, 150 nm, 160 nm, 170 nm, 180 nm, 190 nm, or 200 nm, or less.

A sample may be a biological sample. A may be derived from a biological sample. A biological sample may be, for example, a blood sample, a serum sample, a plasma sample, a saliva sample, a stool sample, a sputum sample, a urine sample, a semen sample, a transvaginal fluid sample, a cerebrospinal fluid sample, a sweat sample, a cell sample, and a tissue sample. A biological sample may be a fluid sample. A fluid sample may be blood or plasma. The sample may be from, or derived from, an animal. The sample may be from, or derived from, a mammal. The sample may be from, or derived from, plant. The sample may be from, or derived from, a human. A sample may comprise nucleic acids.

### Nucleic Acid Targets

A nucleic acid target of the present disclosure may be from a sample. A biological sample may be a sample from, or derived from, a subject. A sample may comprise any number of macromolecules, for example, cellular macromolecules. A sample may comprise a plurality of cells. A sample may be a tissue sample, such as a biopsy, core biopsy, needle aspirate, or fine needle aspirate. The sample may be a tumor sample. A sample may be a fluid sample, such as a blood sample, plasma sample, urine sample, or saliva sample. A sample may be a skin sample. A biological sample may be a cheek swab. A sample may be a plasma or serum sample. A sample may comprise one or more cells. The one or more cells may be from, or derived from a tumor. A biological sample may be, for example, blood, plasma, serum, urine, saliva, mucosal excretions, sputum, stool or tears.

A nucleic acid target may be from, or derived from one or more cells. A nucleic acid target may comprise deoxyribonucleic acid (DNA). DNA may be any kind of DNA, including genomic DNA. A nucleic acid target may be viral DNA. A nucleic acid target may comprise ribonucleic acid (RNA). RNA may be any kind of RNA, including messenger RNA, transfer RNA, ribosomal RNA, and microRNA. RNA may be viral RNA. The nucleic acids may comprise a human genomic sequence. The nucleic acids may comprise an animal genomic sequence. The nucleic acids may comprise a plant genomic sequence. The nucleic acids may comprise a fungal genomic sequence. The nucleic acids may comprise an archaeal genomic sequence. The nucleic acids may comprise a pathogen associated sequence. The nucleic acid may comprise a wild type sequence. The nucleic acid may comprise a variant sequence.

The one or more target nucleic acids may be of any length. A target nucleic acid may be, for example, up to 1, 2, 3, 4, 5, 10, 20, 50, 100, 500, 1000, 5000, 10000, 50000, or 100000 nucleotides, or more.

In some instances, a target nucleic acid may comprise a gene or a portion thereof. A nucleic acid target may comprise a gene whose detection may be useful in diagnosing one or more diseases. A gene may be a viral gene or bacterial gene whose detection may be useful in identifying the presence or absence of a pathogen in a subject. In some cases, the methods of the present disclosure are useful in detecting the presence or absence or one or more infectious agents (e.g., viruses, bacteria, fungi) in a subject. The nucleic acid targets may be a human gene.

The nucleic acid targets may be associated with a disease, such as cancer. The nucleic acid targets may be associated with a degenerative disease. The nucleic acid target may be a nucleic acid from, or derived from, an infectious agent. For example, the nucleic acid target may comprise a sequence from, or derived from, human papilloma virus (HPV) gene. The nucleic acid target may comprise a sequence that presence of HPV in a subject. The nucleic acid target may comprise a sequence from a L1 capsid gene of HPV, L2 capsid gene of HPV, E6 gene of HPV, E7 gene of HPV, and fragments thereof. The nucleic acid targets may encode for proteins or polypeptides. For example, the nucleic acid targets may encode for the L1 capsid protein of HPV, L2 capsid protein of HPV, E6 protein of HPV, E7 protein of HPV, or fragments thereof. The nucleic acid target may comprise a sequence from, or derived from, a Salmonella species. The nucleic acid target may comprise a sequence from, or derived from, Salmonella enterica. The nucleic acid target may comprise a sequence from, or derived from, different species or serovars of Salmonella. For example, the nucleic acid target may comprise a sequence from, or derived from, Salmonella typhimurium, Salmonella enteritidis, Salmonella gallinarum; or Salmonella pullorum. The nucleic acid target may comprise a sequence that is shared between two or more different species or serovars of Salmonella. For example, the nucleic acid target may comprise a sequence shared by Salmonella gallinarum and Salmonella pullorum. In another example, the nucleic acid may comprise a sequence shared between multiples members of the Salmonella genus or multiple strains of Salmonella enterica. For example, the one or more oligonucleotides may bind to that is present is multiple strains of Salmonella enterica and therefore indicate the presence of at least on strain of Salmonella.

In some cases, the methods may be performed by using the compositions as disclosed elsewhere herein. The methods may be used to perform a reaction. The reaction may comprise a hybridization reaction. For example, the composition may comprise a nucleic acid and hybridize with another nucleic acid. The methods may comprise inducing or causing aggregation of one or more nanoparticles. The methods may comprise inducing or causing the formation of a nanoparticle matrix of one or more nanoparticles. The methods may comprise the addition of a solution. The methods may comprise the addition of a condensation agent. The solution (e.g., a condensation solution) and/or condensation agent may cause the aggregation of nanoparticles. For example, the presence of salts may induce aggregation of the nanoparticle. The solution may cause the generation of a nanoparticle matrix. The aggregation or generation of a nanoparticle matrix may be dependent on the structure or molecules associated with the nanoparticle. The nanoparticles may comprise one or more oligonucleotides that may interact to form aggregates or matrices. In the presence of a molecule that binds to the one or more oligonucleotides, (such as a complementary sequence), the aggregation or matrix formation may be prevented or inhibited. The condensation agent may comprise magnesium chloride.

The generation of aggregates of the nanoparticles as compared to the generation of a nanoparticle matrix can be detected and used for detection of the one or more target nucleic acid. A solution may be added to a mixture that cause the nanoparticles to aggregate or alternatively form a nanoparticle matrix. The formation of the aggregate versus the formation of the nanoparticle matrix may be dependent on the presence of the target nucleic acid. Without the presence of the target nucleic acids, the nanoparticle may form aggregates, whereas in the presence of the target nucleic acids the nanoparticles may form a matrix. The aggregates and matrices may have different optical properties and may therefore be detectable and distinguishable based on these optical properties. As the aggregation or matrix formation is dependent on the presence of the target nucleic acid, the detection of the aggregate or the matrix may indicate the presence of the target nucleic acid.

The methods may comprise detecting or determining an optical parameter of solution or composition. The detecting or determining may comprising the use of a sensor for detection of a wavelength. The detecting or determining may comprise the use of a camera. The detecting or determination may comprise image analysis techniques. The optical parameter may comprise an absorbance, transmission, scattering, or reflection of a light or other wave at a wavelength or a range of wavelengths. The optical parameter may comprise a luminosity parameter (e.g., brightness of a color), a saturation parameter (e.g., intensity of a color), or a tonality parameter (e.g., shade of a color). The determining may comprise the use of color spacing analysis. Optical parameters may be binned based on a wavelength or range or wavelengths. For example, an amount of red or green of a color may be parameterized. Based on color theory, a color may not be red and green at the same time, allowing a singly parameter to be generated based on a red-green scale. The parameterization may assign a value to a redness or greenness of a value, wherein the amount of red is parameterized as a positive number and the amount of green. In the same vein, an amount of yellow or blue of a color may be parameterized. Analysis of the red-green and the yellow-blue may indicate a certain parameterized color, which may be used for additional analysis. A luminosity or brightness of a solution may also be parameterized. The optical parameter may be an optical density, for example, an optical density at 520 nm or 560 nm. By observing the optical density of the samples, the samples may be called as a positive or negative. A negative control sample, biocontrol sample, or positive control may also be used to calibrate the method or allow for normalization or referencing of a sample. For example, an optical parameter may be observed to be significantly (e.g., statistically significantly) lower or higher from a negative control, This significant difference may be used to determine a sample as a negative or positive sample. Various wavelengths may be detected using the methods of the disclosure. For example, detection can be performed using wavelengths of at about 300 nm, 305 nm, 310 nm, 315 nm, 320 nm, 325 nm, 330 nm, 335 nm, 340 nm, 345 nm, 350 nm, 355 nm, 360 nm, 365 nm, 370 nm, 375, nm, 380 nm, 385 nm, 390 nm, 395 nm, 400 nm, 405 nm, 410 nm, 415 nm, 420 nm, 425 nm, 430 nm, 435 nm, 440 nm, 445 nm, 450 nm, 455 nm, 460 nm, 465 nm, 470 nm, 475, nm, 480 nm, 485 nm, 490 nm, 495 nm, 500 nm, 505 nm, 510 nm, 515 nm, 520 nm, 525 nm, 530 nm, 535 nm, 540 nm, 545 nm, 550 nm, 555 nm, 560 nm, 565 nm, 570 nm, 575, nm, 580 nm, 585 nm, 590 nm, 595 nm, 600 nm, 605 nm, 610 nm, 615 nm, 620 nm, 625 nm, 630 nm, 635 nm, 640 nm, 645 nm, 650 nm, 655 nm, 660 nm, 665 nm, 670 nm, 675, nm, 680 nm, 685 nm, 690 nm, 695 nm, 700 nm, 705 nm, 710 nm, 715 nm, 720 nm, 725 nm, 730 nm, 735 nm, 740 nm, 745 nm, 750 nm, 755 nm, 760 nm, 765 nm, 770 nm, 775, nm, 780 nm, 785 nm, 790 nm, 795 nm, 800 nm, 805 nm, 810 nm, 815 nm, 820 nm, 825 nm, 830 nm, 835 nm, 840 nm, 845 nm, 850 nm, 855 nm, 860 nm, 865 nm, 870 nm, 875, nm, 880 nm, 885 nm, 890 nm, 895 nm, or 900 nm. For example, detection can be performed using wavelengths of at least 300 nm, 305 nm, 310 nm, 315 nm, 320 nm, 325 nm, 330 nm, 335 nm, 340 nm, 345 nm, 350 nm, 355 nm, 360 nm, 365 nm, 370 nm, 375, nm, 380 nm, 385 nm, 390 nm, 395 nm, 400 nm, 405 nm, 410 nm, 415 nm, 420 nm, 425 nm, 430 nm, 435 nm, 440 nm, 445 nm, 450 nm, 455 nm, 460 nm, 465 nm, 470 nm, 475, nm, 480 nm, 485 nm, 490 nm, 495 nm, 500 nm, 505 nm, 510 nm, 515 nm, 520 nm, 525 nm, 530 nm, 535 nm, 540 nm, 545 nm, 550 nm, 555 nm, 560 nm, 565 nm, 570 nm, 575, nm, 580 nm, 585 nm, 590 nm, 595 nm, 600 nm, 605 nm, 610 nm, 615 nm, 620 nm, 625 nm, 630 nm, 635 nm, 640 nm, 645 nm, 650 nm, 655 nm, 660 nm, 665 nm, 670 nm, 675, nm, 680 nm, 685 nm, 690 nm, 695 nm, 700 nm, 705 nm, 710 nm, 715 nm, 720 nm, 725 nm, 730 nm, 735 nm, 740 nm, 745 nm, 750 nm, 755 nm, 760 nm, 765 nm, 770 nm, 775, nm, 780 nm, 785 nm, 790 nm, 795 nm, 800 nm, 805 nm, 810 nm, 815 nm, 820 nm, 825 nm, 830 nm, 835 nm, 840 nm, 845 nm, 850 nm, 855 nm, 860 nm, 865 nm, 870 nm, 875, nm, 880 nm, 885 nm, 890 nm, 895 nm, or 900 nm, or more. For example, detection can be performed using wavelengths of no more than 300 nm, 305 nm, 310 nm, 315 nm, 320 nm, 325 nm, 330 nm, 335 nm, 340 nm, 345 nm, 350 nm, 355 nm, 360 nm, 365 nm, 370 nm, 375, nm, 380 nm, 385 nm, 390 nm, 395 nm, 400 nm, 405 nm, 410 nm, 415 nm, 420 nm, 425 nm, 430 nm, 435 nm, 440 nm, 445 nm, 450 nm, 455 nm, 460 nm, 465 nm, 470 nm, 475, nm, 480 nm, 485 nm, 490 nm, 495 nm, 500 nm, 505 nm, 510 nm, 515 nm, 520 nm, 525 nm, 530 nm, 535 nm, 540 nm, 545 nm, 550 nm, 555 nm, 560 nm, 565 nm, 570 nm, 575, nm, 580 nm, 585 nm, 590 nm, 595 nm, 600 nm, 605 nm, 610 nm, 615 nm, 620 nm, 625 nm, 630 nm, 635 nm, 640 nm, 645 nm, 650 nm, 655 nm, 660 nm, 665 nm, 670 nm, 675, nm, 680 nm, 685 nm, 690 nm, 695 nm, 700 nm, 705 nm, 710 nm, 715 nm, 720 nm, 725 nm, 730 nm, 735 nm, 740 nm, 745 nm, 750 nm, 755 nm, 760 nm, 765 nm, 770 nm, 775, nm, 780 nm, 785 nm, 790 nm, 795 nm, 800 nm, 805 nm, 810 nm, 815 nm, 820 nm, 825 nm, 830 nm, 835 nm, 840 nm, 845 nm, 850 nm, 855 nm, 860 nm, 865 nm, 870 nm, 875, nm, 880 nm, 885 nm, 890 nm, 895 nm, or 900 nm, or less. Detection can be performed by obtaining a spectra from 300 nm to 900 nm, or any subset of wavelength ranges. For example, detection can be performed from 450 nm to 700 nm.

The detection may be performed using a plate reader, spectrophotometer, or other instrument capable of detection of light or UV waves. For example, the instrument may comprise a detector that can quantify the amount of light received. The instrument can use a monochromator to direct a specific wavelength to the sample or observe a given wavelength from a sample.

The methods may comprise comparing an optical parameter of a test solution to a reference or control solution. The optical parameters of the test solution and the reference solution may be determined and may be compared. Based on the comparison of the test solution and reference solution, a detection of a target nucleic acid may be determined. For example, the reference solution may be a positive control solution and comprise the target nucleic acid. This reference solution may comprise an optical parameter. The test solution may be also analyzed if the solution comprises the target nucleic acid, the optical parameters of the test solution and the reference solution may be the same (or be substantially similar). A reference solution may also be a negative control type solution wherein the target nucleic acid is absent or another known sequence that is not the target sequence is present. The optical parameters of the test solution and reference solution may be compared and determinations may be generated based on the similarity of the optical parameters.

The aggregated nanoparticles or nanoparticle matrix may comprise an optical parameter that is not the same as the one or more nanoparticles in solution. Because the optical parameters may be different when comparing an aggregated nanoparticle (or nanoparticle matrix) to a nanoparticle in solution, it may be possible to detect the presence of aggregated nanoparticles or nanoparticles matrix and distinguish a solution with aggregated particles (or nanoparticle matrix) from a solution with non-aggregated nanoparticles or single nanoparticles in solution. The detection of a target nucleic acid may use this difference to determine if a target nucleic acid is present. The target nucleic acid may inhibit the formation of aggregates or matrices, such that the lack of aggregates or matrices may indicated that the target nucleic acid is present.

For example, a biosensor homogeneous dispersion (AuNP-ASO; no target nucleic acid; no condensation agent): can be characterized as maximum distance from AuNP-ASO, observing the minimum agglomeration of particles. Reactivity can be visualized by the partial change in the reddish color. A positive heterogeneous dispersion (AuNP-ASO+target RNA/DNA+MgCl₂): can be characterized as controlled distancing of AuNP-ASO, through bridges formed in the recognition of genetic material (RNA/DNA). The annealing distance may comprise a minimum number of bases (60±10 nucleotides). Partial agglomeration of particles in the presence of MgCl₂, may be observed and associated with an opaque gray/purple color visualization. A negative heterogeneous dispersion (AuNP-ASO+MgCl₂): it can be characterized as high agglomeration of AuNP-ASO in the presence of MgCl₂, associated with the visualization of the change from reddish to gray/translucent blue. Based on the color of the solution, which is based at least on the agglomeration, the different dispersion pattern can be differentiated and the presence of a target nucleic acid can be determined.

The method may determine the presence or absence of the one or more target nucleic acids in the bodily sample at a sensitivity of at least 90%, 91%, 92%, 93%, 94%, 95%, 96%, 97%, 98%, or 99%. The method may determine the presence or absence of the one or more target nucleic acids in the bodily sample at a sensitivity of at least 90%, 91%, 92%, 93%, 94%, 95%, 96%, 97%, 98%, or 99%. The method may determine the presence or absence of the one or more target nucleic acids in the bodily sample at a specificity of at least 95%, 96%, 97%, 98%, or 99%. The method determines the presence or absence of the one or more target nucleic acids in the bodily sample at a precision of at least 90%, 91%, 92%, 93%, 94%, 95%, 96%, 97%, 98%, or 99%.

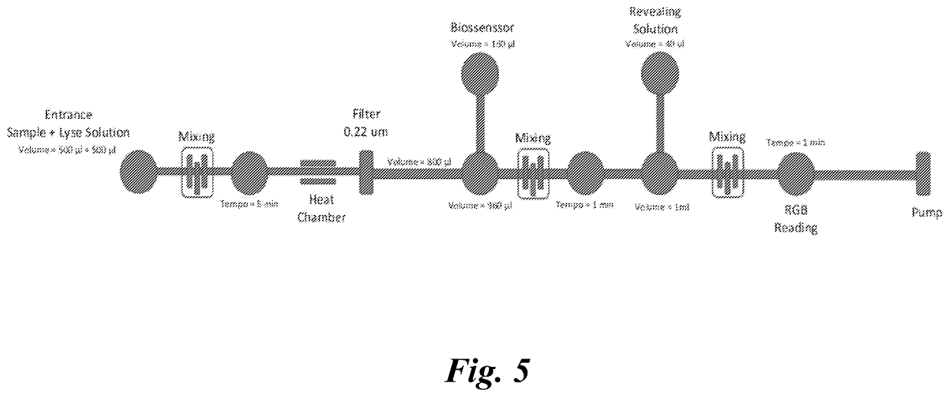

The method may be performed using devices that allow the method to be performed. FIG. 5 shows an example device. An entrance to the device is available for adding a sample and a lyse solution. This solution is then allowed to flow into a mixing module, heat chamber, and filter such that the sample solution contains extracted nucleic acids. The sample is allowed to flow into a pool in which a biosensor (e.g. AuNP comprising oligonucleotides) is added. This is allowed to mix in a mixing module and then flowed to a new pool for a revealing solution to be added (e.g., a condensation solution). This solution is then mixed in a mixing module and then flowed into a new pool to perform an optical reading such as a RGB reading or determination of another optical parameter, such as one described elsewhere herein.

The methods may be performed in a plate comprising one or more wells. For example, the methods may use a 96 well plate. Multiple assay may be performed simultaneously, for example, where a given sample is present is each well. The wells can then be analyzed using plate reader and an output can be provided for each sample. As such, the assays allow for rapid and efficient multiplexing which can reduce wait time and improve throughput. The methods may also be performed using a cuvette or tube, or other container. For example, the methods may be performed by mixing samples in tubes (e.g., centrifuge or microcentrifuge tubes). The samples may then be added to a cuvette to be read via a spectrophotometer or other instrument capable to detecting optical properties.

The ability of the methods and compositions to identify the presence of a target nucleic acid can be measured in terms of the accuracy of the assay, the sensitivity of the assay, the specificity of the assay, the positive predictive value (PPV) of the assay, the negative predictive value (NPV) of the assay, or the “Area Under a Curve” (AUC), for example, the area under a Receiver Operating Characteristic (ROC) curve. As used herein, accuracy is a measure of the fraction of misclassified samples. Accuracy may be calculated as the total number of correctly classified samples divided by the total number of samples, e.g., in a test population. Sensitivity is a measure of the “true positives” that are predicted by a test to be positive, and may be calculated as the number of correctly identified cancer samples divided by the total number of cancer samples. Specificity is a measure of the “true negatives” that are predicted by a test to be negative, and may be calculated as the number of correctly identified normal samples divided by the total number of normal samples. AUC is a measure of the area under a Receiver Operating Characteristic curve, which is a plot of sensitivity vs. the false positive rate (1-specificity). The greater the AUC, the more powerful the predictive value of the test. In some embodiments, the methods can identify the presence of a target nucleic acids at an area under curve (AUC) of at least about 0.9. In some embodiments, methods can identify the presence of a target nucleic acids at an area under curve (AUC) of at least about 0.91, 0.92, 0.93, 0.94, 0.95, 0.96, 0.97, 0.98, 0.99, or higher. In some embodiments, methods can identify the presence of a target nucleic acids at an area under curve (AUC) of at least about 0.50, at least about 0.55, at least about 0.60, at least about 0.65, at least about 0.70, at least about 0.75, at least about 0.80, at least about 0.85, at least about 0.90, at least about 0.95, or more. Other useful measures of the utility of a test include the “positive predictive value,” which is the percentage of actual positives who test as positives, and the “negative predictive value,” which is the percentage of actual negatives who test as negatives. In some embodiments, methods can identify the presence of a target nucleic acids at a positive predictive value of at least about 50%, at least about 55%, at least about 60%, at least about 65%, at least about 70%, at least about 75%, at least about 80%, at least about 85%, at least about 90%, at least about 95%, or more In some embodiments, methods can identify the presence of a target nucleic acids at a negative predictive value of at least about 50%, at least about 55%, at least about 60%, at least about 65%, at least about 70%, at least about 75%, at least about 80%, at least about 85%, at least about 90%, at least about 95%, or more. In some embodiments, methods described herein show an accuracy of at least about 75%, e.g., an accuracy of at least about 75%, about 80%, about 85%, about 90%, about 95%, about 97%, about 99% or about 100%. For example, the methods can identify the presence of a target nucleic acids at an area at least about 50%, at least about 55%, at least about 60%, at least about 65%, at least about 70%, at least about 75%, at least about 80%, at least about 85%, at least about 90%, at least about 95%, or more. In other embodiments, methods can identify the presence of a target nucleic acids at a specificity of at least about 75%, e.g., a specificity of at least about 75%, about 80%, about 85%, about 90%, about 95%, about 97%, about 99% or about 100%. For example, methods can identify the presence of a target nucleic acids at a specificity of at least about 50%, at least about 55%, at least about 60%, at least about 65%, at least about 70%, at least about 75%, at least about 80%, at least about 85%, at least about 90%, at least about 95%, or more. In some embodiments, methods can identify the presence of a target nucleic acids at least about 75%, e.g., a sensitivity of at least about 75%, about 80%, about 85%, about 90%, about 95%, about 97%, about 99% or about 100%. For example, methods can identify the presence of a target nucleic acids at a sensitivity of at least about 50%, at least about 55%, at least about 60%, at least about 65%, at least about 70%, at least about 75%, at least about 80%, at least about 85%, at least about 90%, at least about 95%, or more. In other embodiments, methods can identify the presence of a target nucleic acids at a specificity and sensitivity of at least about 75% each, e.g., a specificity and sensitivity of at least about 75%, about 80%, about 85%, about 90%, about 95%, about 97%, about 99% or about 100% (for example, a specificity of at least about 80% and sensitivity of at least about 80%, or for example, a specificity of at least about 80% and sensitivity of at least about 95%).

The methods of the disclosure may be performed in a short period of time and may be faster than other methods that have similar metrics of accuracy. For example, the methods of the disclosure may be performed in no more than, 60 minutes, 55 minutes, 50 minutes, 45 minutes, 40 minutes, 35 minutes, 30 minutes, 25 minutes, 20 minutes, 15 minutes, 10 minutes, 5 minutes, or less. The methods, starting with pre-assembled nanoparticles (e.g., one or more nanoparticles assembled to one or more oligonucleotides), may be performed in less than 60 minutes. The methods, starting with pre-assembled nanoparticles (e.g., one or more nanoparticles assembled to one or more oligonucleotides), may be performed in no more than 50 minutes, 45 minutes, 40 minutes, 35 minutes, 30 minutes, 25 minutes, 20 minutes, 15 minutes, 10 minutes, 5 minutes, or less. The methods, starting with one or more nanoparticles that are not assembled to one or more oligonucleotides, may be performed in no more than, 60 minutes, 55 minutes, 50 minutes, 45 minutes, 40 minutes, 35 minutes, 30 minutes, 25 minutes, 20 minutes, 15 minutes, 10 minutes, 5 minutes, or less.

### Kits

The present disclosure provides kits for performing the methods of the disclosures. The present disclosure also provides kits comprising the compositions described in this disclosure. The kits may comprise nanoparticles and oligonucleotides as described elsewhere herein. For example, the kits may comprise a nanoparticle assembled to one or more oligonucleotides. The kits may comprise a condensation solution. The kits may comprise tubes comprising the nanoparticles.

The kits may comprise instructions for using any of the foregoing in the methods described herein. The kits may comprise solutions or other components that may be used a standard, a negative or positive control. For example, the kits may comprise a standard that may be used as a baseline for color analysis or optical parameter determination. The kits may comprise a swab or other implement for collecting a sample for a subject. The kits may comprise a device for adding solutions to or for otherwise running the methods of the disclosure.

### Computer Control Systems

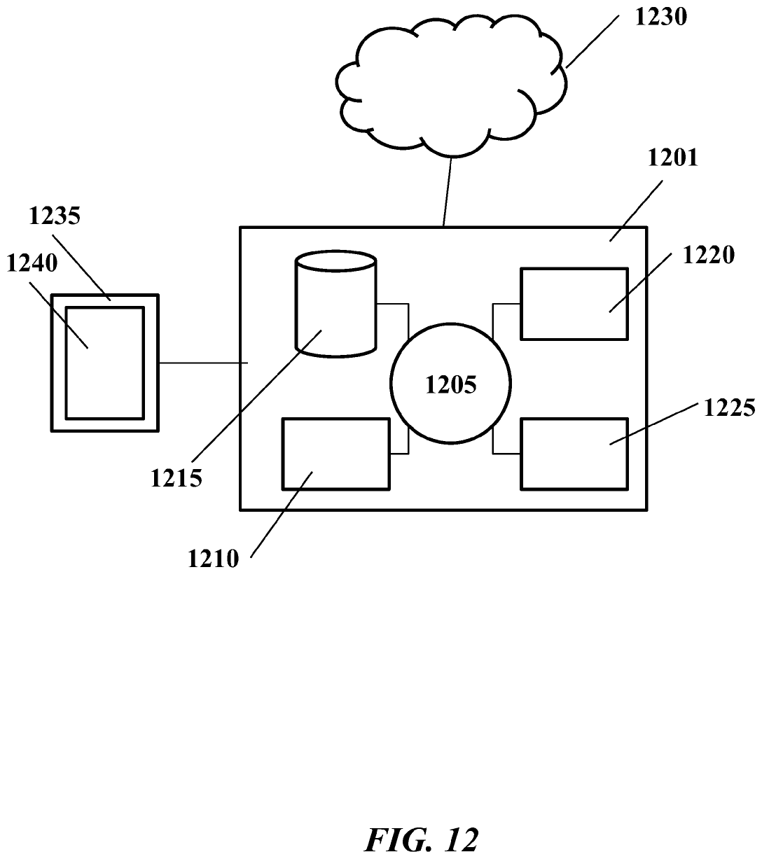

The present disclosure provides computer control systems that are programmed to implement methods of the disclosure. FIG. 12 shows a computer system 1201 that is programmed or otherwise configured to perform parts of the methods disclosed elsewhere herein. The computer system 1201 can regulate various aspects of the present disclosure, such as, for example process images of a sample, determine and process optical parameter, receive images from a user, process images such that the color and color differentials of the sample and reference colors can be identified, process images using an edge detector algorithm, output a result for the user as to the presence of a nucleic acid. The computer system 1201 can be an electronic device of a user or a computer system that is remotely located with respect to the electronic device. The electronic device can be a mobile electronic device.

The computer system 1201 includes a central processing unit (CPU, also “processor” and “computer processor” herein) 1205, which can be a single core or multi core processor, or a plurality of processors for parallel processing. The computer system 1201 also includes memory or memory location 1210 (e.g., random-access memory, read-only memory, flash memory), electronic storage unit 1215 (e.g., hard disk), communication interface 1220 (e.g., network adapter) for communicating with one or more other systems, and peripheral devices 1225, such as cache, other memory, data storage and/or electronic display adapters. The memory 1210, storage unit 1215, interface 1220 and peripheral devices 1225 are in communication with the CPU 1205 through a communication bus (solid lines), such as a motherboard. The storage unit 1215 can be a data storage unit (or data repository) for storing data. The computer system 1201 can be operatively coupled to a computer network (“network”) 1230 with the aid of the communication interface 1220. The network 1230 can be the Internet, an internet and/or extranet, or an intranet and/or extranet that is in communication with the Internet. The network 1230 in some cases is a telecommunication and/or data network. The network 1230 can include one or more computer servers, which can enable distributed computing, such as cloud computing. The network 1230, in some cases with the aid of the computer system 1201, can implement a peer-to-peer network, which may enable devices coupled to the computer system 1201 to behave as a client or a server.

The CPU 1205 can execute a sequence of machine-readable instructions, which can be embodied in a program or software. The instructions may be stored in a memory location, such as the memory 1210. The instructions can be directed to the CPU 1205, which can subsequently program or otherwise configure the CPU 1205 to implement methods of the present disclosure. Examples of operations performed by the CPU 1205 can include fetch, decode, execute, and writeback.

The CPU 1205 can be part of a circuit, such as an integrated circuit. One or more other components of the system 1201 can be included in the circuit. In some cases, the circuit is an application specific integrated circuit (ASIC).

The storage unit 1215 can store files, such as drivers, libraries and saved programs. The storage unit 1215 can store user data, e.g., user preferences and user programs. The computer system 1201 in some cases can include one or more additional data storage units that are external to the computer system 1201, such as located on a remote server that is in communication with the computer system 1201 through an intranet or the Internet.

The computer system 1201 can communicate with one or more remote computer systems through the network 1230. For instance, the computer system 1201 can communicate with a remote computer system of a user (e.g. a patient, medical provider, test administrator). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 1201 via the network 1230.

Methods as described herein can be implemented by way of machine (e.g., computer processor) executable code stored on an electronic storage location of the computer system 1201, such as, for example, on the memory 1210 or electronic storage unit 1215. The machine executable or machine readable code can be provided in the form of software. During use, the code can be executed by the processor 1205. In some cases, the code can be retrieved from the storage unit 1215 and stored on the memory 1210 for ready access by the processor 1205. In some situations, the electronic storage unit 1215 can be precluded, and machine-executable instructions are stored on memory 1210.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 1201, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 1201 can include or be in communication with an electronic display 1235 that comprises a user interface (UI) 1240 for providing, for example, results of the methods, optical parameters or images of the solutions. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 1205. The algorithm can, for example, determine optical parameters of a solution via image analysis, perform comparisons of optical parameters, or normalize optical parameters against a baseline or control.

### List of Embodiments

Embodiment 1. A method for processing or analyzing a sample, the method comprising:
(a) contacting the sample with a composition that comprises one or more nanoparticles assembled with one or more oligonucleotides to provide a test composition, wherein the one or more oligonucleotides hybridize to one or more target nucleic acid, if present, in the sample;
(b) in the presence of the one or more target nucleic acids, forming a nanoparticle matrix from the one or more nanoparticles hybridized to the one or more target nucleic acids;
(c) determining an optical parameter of the test composition that is indicative of the presence or absence of the one or more nucleic acids in the sample.

Embodiment 2. The method of embodiment 1, wherein the optical parameter is determined by a color spacing analysis.

Embodiment 3. The method of embodiments 1 or embodiment 2, wherein the optical parameter comprises absorbance, transmission, scattering, or reflection of a light at a wavelength or a range of wavelengths.

Embodiment 4. The method of any one of embodiments 1-3, wherein the optical parameter comprises a luminosity parameter (e.g., brightness of a color), a saturation parameter (e.g., intensity of a color), or a tonality parameter (e.g., shade of a color).

Embodiment 5. The method of any one of embodiments 1-4, wherein (c) further comprises comparing the optical parameter of the test composition with a corresponding optical parameter determined from a corresponding reference composition.

Embodiment 6. The method of any one of embodiments 1-5, wherein method determines the presence or absence of the one or more target nucleic acids in the sample at a sensitivity of at least 90%, 91%, 92%, 93%, 94%, 95%, 96%, 97%, 98%, or 99%.

Embodiment 7. The method of any one of embodiments 1-6, wherein the method determines the presence or absence of the one or more target nucleic acids in the sample at a specificity of at least 90%, 91%, 92%, 93%, 94%, 95%, 96%, 97%, 98%, or 99%.

Embodiment 8. The method of any one of embodiments 1-7, wherein the method determines the presence or absence of the one or more target nucleic acids in the sample at a precision of at least 90%, 91%, 92%, 93%, 94%, 95%, 96%, 97%, 98%, or 99%.

Embodiment 9. The method of any one of embodiments 1-8, wherein the sample is selected from: a blood sample, a serum sample, a plasma sample, a saliva sample, a stool sample, a sputum sample, a urine sample, a semen sample, a transvaginal fluid sample, a cerebrospinal fluid sample, a sweat sample, a cell sample, and a tissue sample.

Embodiment 10. The method of any one of embodiments 1-9, wherein the sample is from a mammal (e.g., a human).

Embodiment 11. The method of any of embodiments 1-10, wherein the sample is from an animal.

Embodiment 12. The method of any one of embodiments 1-11, wherein the sample is from a plant.

Embodiment 13. The method of any one of embodiments 1-12, wherein the sample comprises a lysis solution.

Embodiment 14. The method of any one of embodiments 1-13, wherein (b) comprises contacting the test composition with a nanoparticle condensation agent and/or salt.

Embodiment 15. The method of embodiment 14, wherein the condensation agent comprises magnesium chloride.

Embodiment 16. The method of any one of embodiments 1-15, wherein at least about 40% nucleotides of the one or more oligonucleotides are guanine or cytosine.

Embodiment 17. The method of any one of embodiments 1-15 wherein about 40% to about 60% nucleotides of the one or more oligonucleotides are guanine or cytosine.

Embodiment 18. The method of any one of embodiments 1-17, wherein the one or more oligonucleotides are characterized by a melting temperature (Tm) of at least about 65 degree Celsius (° C.).

Embodiment 19. The method of any one of embodiments 1-17, wherein the one or more oligonucleotides are characterized by a Tm of about 65° C. to about 75° C.

Embodiment 20. The method of any one of embodiments 1-19, wherein the one or more oligonucleotides comprise a conjugating moiety at the 5′-end.

Embodiment 21. The method of embodiment 20, wherein the conjugating moiety is 5′-thiol.

Embodiment 22. The method of embodiment 21, wherein the 5′-thiol comprise a thioalkyl, such as thiohexyl.

Embodiment 23. The method of any one of embodiments 1-22, wherein a nanoparticle of the one or more nanoparticles is assembled with one, two, three, four, five, or six oligonucleotide(s).

Embodiment 24. The method of any one of embodiments 1-23, wherein the one or more nanoparticles are each (e.g., independently) assembled with one, two, three, four, five, or six oligonucleotide(s).

Embodiment 25. The method of any one of embodiments 1-24, wherein the one or more nanoparticles comprise gold.

Embodiment 26. The method of any one of embodiments 1-25, wherein the one or more nanoparticles are characterized by an average size of about 10 nanometers (nm) to about 200 nm.

Embodiment 27. The method of any one of embodiments 1-26, wherein the one or more oligonucleotides are 16 to 24 nucleotides long.

Embodiment 28. The method of any one of embodiments 1-27, wherein the one or more oligonucleotides are associated with the one or more target nucleic acid such that a distance between two adjacent nanoparticles of the one or more nanoparticles correspond to about 50 to about 70 nucleotides.

Embodiment 29. The method of any one of embodiments 1-28, wherein the one or more oligonucleotides comprises two oligonucleotides, wherein the first oligonucleotide hybridizes to a first region of the target nucleic acid, and the second oligonucleotide hybridizes to a second region of the target nucleic acid.

Embodiment 30. The method of embodiment 29, wherein the distance between the first region and the second region of the target nucleic acid is about 50 to 70 nucleotides.

Embodiment 31. The method of any one of embodiments 1-30, wherein the one or more oligonucleotides hybridize to 10 to 30 nucleotides of the one or more target nucleic acids.

Embodiment 32. The method of any one of embodiments 1-31, wherein, in the absence of one or more target nucleic acids, the one or more nanoparticles form aggregates.

Embodiment 33. The method of any one of embodiments 1-32, wherein the one or more target nucleic acids are from one or more viruses or one or more bacteria.

Embodiment 34. The method of embodiment 33, wherein the one or more target nucleic acids are not from a coronavirus.

Embodiment 35. The method of embodiment 33, wherein the one or more target nucleic acids are not from SARS-CoV-2 or one or more variants thereof.

Embodiment 36. The method of any one of embodiments 33-35, wherein the one or more viruses comprises an influenza virus, or a human papilloma virus.

Embodiment 37. The method of any one of embodiments 33-36, wherein the one or more bacteria comprises a Salmonella.

Embodiment 38. The method of embodiment 37, wherein the Salmonella comprises a Salmonella enterica.

Embodiment 39. The method of embodiment 37, wherein the Salmonella comprises one or more Salmonella strains or serovars.

Embodiment 40. The method of embodiment 39, wherein the one or more Salmonella strains or serovars comprises one or more members selected from the group consisting of Salmonella typhimurium, Salmonella enteritidis, Salmonella gallinarum and Salmonella pullorum.

Embodiment 41. The method of any one of embodiments 1-40, wherein the one or more target nucleic acids are associated with one or more diseases or conditions.

Embodiment 42. The method of embodiment 41, wherein the one or more diseases or conditions comprise an infectious disease, a cancer, or a degenerative disease.

Embodiment 43. The method of any one of embodiments 1-42 wherein the one or more target nucleic acids encode for a polypeptide or protein.

Embodiment 44. The method of any one of embodiments 1-43, wherein the one or more target nucleic acids comprise a DNA or RNA.

Embodiment 45. The method of embodiment 44, wherein the DNA is a genomic DNA.

Embodiment 46. The method of embodiment 44, wherein the RNA is a genomic RNA.

Embodiment 47. The method of embodiment 44, wherein the RNA is a double stranded RNA or a single stranded RNA.

Embodiment 48. The method of embodiment 44, wherein the RNA is a double stranded DNA or a single stranded DNA.

Embodiment 49. The method of any one of embodiments 1-48, wherein the one or more target nucleic acid are from human papillomavirus (HPV) or one or more variants thereof.

Embodiment 50. The method of any one of embodiments 1-49, wherein the one or more target nucleic acid comprise one or more members selected from: L1 capsid protein of HPV, L2 capsid protein of HPV, E6 protein of HPV, E7 protein of HPV, and fragments of either thereof

Embodiment 51. The method of any one of embodiments 1-50, wherein the one or more target nucleic acids are from a Salmonella.

Embodiment 52. The method of embodiment 51, wherein the Salmonella comprises a Salmonella enterica.

Embodiment 53. The method of embodiment 51, wherein the Salmonella comprises one or more Salmonella strains or serovars.

Embodiment 54. The method of embodiment 53, wherein the one or more Salmonella strains or serovars comprises one or more members selected from the group consisting of Salmonella typhimurium, Salmonella enteritidis, Salmonella gallinarum and Salmonella pullorum.

Embodiment 55. The method of any one of embodiments 1-54, wherein the one or more oligonucleotides comprise a sequence selected from SEQ ID NOS: 1-18.

Embodiment 56. A composition for detecting one or more target nucleic acid, the composition comprising:
one or more nanoparticles assembled with one or more oligonucleotides, wherein the one or more oligonucleotides are complementary to the one or more target nucleic acid,
wherein, in the presence of the one or more target nucleic acid, the one or more nanoparticles form a nanoparticle matrix, wherein the nanoparticle matrix comprises a different optical parameter compared to a solution comprising corresponding nanoparticles that are not in a nanoparticle matrix.

Embodiment 57. A composition for detecting a target nucleic acid, the composition comprising:
one or more nanoparticles assembled with one or more oligonucleotides, wherein a first oligonucleotide of the one or more oligonucleotides is complementary to said target nucleic acid, and wherein a second oligonucleotide of the one or more oligonucleotides is complementary to said target nucleic acid at a second sequence, wherein the one or more nanoparticles comprise gold,
wherein, in the presence of the one or more target nucleic acid, the one or more nanoparticles form a nanoparticle matrix.

Embodiment 58. The composition of any one of embodiments 56 or 57, wherein at least about 40% (e.g., about 40% to about 60%)) nucleotides of the one or more oligonucleotides are guanine or cytosine.

Embodiment 59. The composition of any one of embodiments 56-58, wherein the one or more oligonucleotides are characterized by a melting temperature (Tm) of at least about 65 degree Celsius (C) (e.g., of about 65° C. to about 75° C.).

Embodiment 60. The composition of any one of embodiments 56-59, wherein the one or more oligonucleotides comprise a conjugating moiety at the 5′-end.

Embodiment 61. The composition of embodiment 60, wherein the conjugating moiety is 5′-thiol.

Embodiment 62. The composition of embodiment 61, wherein the 5′ thiol is a thioalkyl group.

Embodiment 63. The composition of embodiment 62, wherein the thioalkyl is a thiohexyl group.

Embodiment 64. The composition of any one of embodiments 56-63, wherein a nanoparticle of the one or more nanoparticles is assembled with one, two, three, four, five, or six oligonucleotide(s).

Embodiment 65. The composition of any one of embodiments 56-64, wherein the one or more nanoparticles are each (e.g., independently) assembled with one, two, three, four, five, or six oligonucleotide(s).

Embodiment 66. The composition of any one of embodiments 56-65, wherein the one or more nanoparticles comprise gold.

Embodiment 67. The composition of any one of embodiments 56-66, wherein the one or more nanoparticles are characterized by an average size of about 10 nanometers (nm) to about 200 nm.

Embodiment 68. The composition of any one of embodiments 56-67, wherein the one or more oligonucleotides are 16 to 24 nucleotides long.

Embodiment 69. The composition of any one of embodiments 56-68, wherein the one or more oligonucleotides are associated with the one or more target nucleic acid such that a distance between two adjacent nanoparticles of the one or more nanoparticles correspond to about 50 to about 70 nucleotides.

Embodiment 70. The composition of any one of embodiments 56-69, wherein the one or more oligonucleotides comprises two oligonucleotides, wherein the first oligonucleotide hybridizes to a first region of the target nucleic acid, and the second oligonucleotide hybridizes to a second region of the target nucleic acid.

Embodiment 71. The composition of embodiment 70, wherein the distance between the first region and the second region of the target nucleic acid is about 50 to 70 nucleotides. Embodiment 72. The composition of any one of embodiments 56-69, wherein the one or more target nucleic acids are from one or more viruses or one or more bacteria. Embodiment 73. The composition of embodiment 72, wherein the one or more target nucleic acids are not from a coronavirus.

Embodiment 74. The composition of embodiment 73, wherein the one or more target nucleic acids are not from SARS-CoV-2 or one or more variants thereof.

Embodiment 75. The composition of any one of embodiments 68-74, wherein the one or more viruses comprises an influenza virus, or a human papilloma virus.

Embodiment 76. The composition of embodiment 73, wherein the one or more bacteria comprises a Salmonella.

Embodiment 77. The composition of embodiment 76, wherein the Salmonella comprises a Salmonella enterica.

Embodiment 78. The composition of embodiment 77, wherein the Salmonella comprises one or more Salmonella strains or serovars.

Embodiment 79. The composition of embodiment 78, wherein the one or more Salmonella strains or serovars comprises one or more members selected from the group consisting of Salmonella typhimurium, Salmonella enteritidis, Salmonella gallinarum and Salmonella pullorum.

Embodiment 80. The composition of any one of embodiments 56-79, wherein the one or more target nucleic acids are associated with one or more diseases or conditions.

Embodiment 81. The composition of embodiment 80, wherein the one or more diseases or conditions comprise an infectious disease, a cancer, or a degenerative disease.

Embodiment 82. The composition of any one of embodiments 56-81, wherein the one or more target nucleic acids encode for a polypeptide or protein.

Embodiment 83. The composition of any one of embodiments 56-82, wherein the one or more target nucleic acids comprise a DNA or RNA.

Embodiment 84. The composition of embodiment 83, wherein the DNA is a genomic DNA.

Embodiment 85. The composition of embodiment 83, wherein the RNA is a genomic RNA.

Embodiment 86. The composition of embodiment 83, wherein the RNA is a double stranded RNA or a single stranded RNA.

Embodiment 87. The composition of embodiment 83, wherein the RNA is a double stranded DNA or a single stranded DNA.

Embodiment 88. The composition of any one of embodiments 56-87, wherein the one or more target nucleic acid are from human papillomavirus (HPV) or one or more variants thereof.

Embodiment 89. The composition of any one of embodiments 56-88, wherein the one or more target nucleic acid comprise one or more members selected from: L1 capsid protein of HPV, L2 capsid protein of HPV, E6 protein of HPV, E7 protein of HPV, and fragments of either thereof.

Embodiment 90. The composition of any one of embodiments 56-89, wherein the one or more target nucleic acids are from a Salmonella.

Embodiment 91. The composition of embodiment 90, wherein the Salmonella comprises a Salmonella enterica.

Embodiment 92. The composition of embodiment 90, wherein the Salmonella comprises one or more Salmonella strains or serovars.

Embodiment 93. The composition of embodiment 92, wherein the one or more Salmonella strains or serovars comprises one or more members selected from the group consisting of Salmonella typhimurium, Salmonella enteritidis, Salmonella gallinarum and Salmonella pullorum.

Embodiment 94. The composition of any one of embodiments 56-93, wherein the one or more oligonucleotides comprise a sequence selected from SEQ ID NOS: 1-18.

Embodiment 95. A kit for identifying the presence of a target nucleic acid, the kit comprising: (i) one or more gold nanoparticles assembled with one or more oligonucleotides, (ii) a condensation solution, (iii) instructions for using said one or more gold nanoparticles assembled with one or more oligonucleotides.

Embodiment 96. A kit for identifying the presence of a target nucleic acid, the kit comprising: (i) the composition of any of embodiments 56 to 94, (ii) a condensation solution, (iii) instructions for using said one or more gold nanoparticles assembled with one or more oligonucleotides.

### EXAMPLES

### Example 1: Generation of the Nanoparticles Comprising Oligonucleotides

Gold nanoparticles comprising antisense oligonucleotides (ASOs) were generated by conjugating the nucleic acids on to the surface of the nanoparticles. The binding of the nucleic acid sequences on the surface of the nanoparticles occur by the covalent bond of the ASOs replacing a stabilizing citrate group. The conjugation step was performed by ligating the oligonucleotide with the gold particles. Each ASO molecule is able to form a covalent bond by the thiol group in only a single gold particle.

First, the gold nanoparticle was combined with ASOs by adding the nanoparticle and ASOs with citric acid and hydrochloric acid. The solution was homogenized by stirring, the conjugation is allowed to occur. Then, the solution was centrifuged to remove free ASO molecules (e.g., ASOs that did not bind to the gold particle). After centrifugation, the supernatant was discarded and the pellet was resuspended with HEPES buffer. The final product does not contain free ASO molecules in order to increase signal sensitivity, and all gold should be bound to ASOs.

The conjugation process was carried out using 2 mL of the suspension of gold nanoparticles (AuNPs) with a concentration of 1 mM of Au atoms and an average size of 20 nm, adding 36.6 μL of ASOs, at a concentration of 50 μM to obtain a ratio of 1 particle to 900 strands of nucleic acid. After 10 minutes of stirring at 350 rpm at room temperature, 10 μL of sodium citrate solution (500 mM, pH 3.0) and 5 μL of hydrochloric acid solution (1 M) was added to adjust the pH to 3.0. After this step, the solution was stirred at 350 rpm for 20 minutes and then centrifuged at 14,000 rpm for 15 minutes. The supernatant was discarded and the pellet was resuspended with 2 mL of HEPES buffer (10 mM).

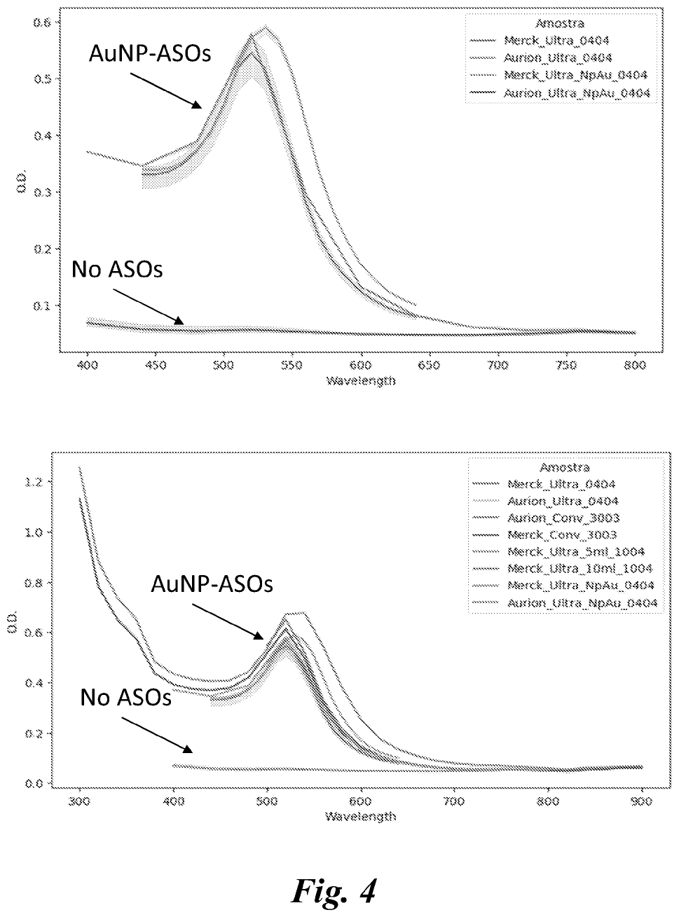

FIG. 4 shows a multiple UV-visible curves of nanoparticles coupled to ASOs. Nanoparticles coupled to ASOs demonstrate a large peak on the UV-visible spectrum, whereas the nanoparticles alone without ASO show no UV-visible. Of note, this can also be observed via the naked eye, where the AuNP-ASO solution is a reddish color, whereas the solution of uncoupled nanoparticles is generally colorless and transparent.

FIG. 3 shows example schematics of the nano particle-ASOs that can be made using this method. As shown, a single nanoparticle may have multiple different oligonucleotides attached (“single-nano multiplex”). Additionally multiple oligonucleotides may be attached to different nanoparticles (“multi-nano multiplex”) where multiple nanoparticles may be attached to oligonucleotides with the same sequence, and may also be attached to oligonucleotides with different sequences.

### Example 2: Detection of Nucleic Acids

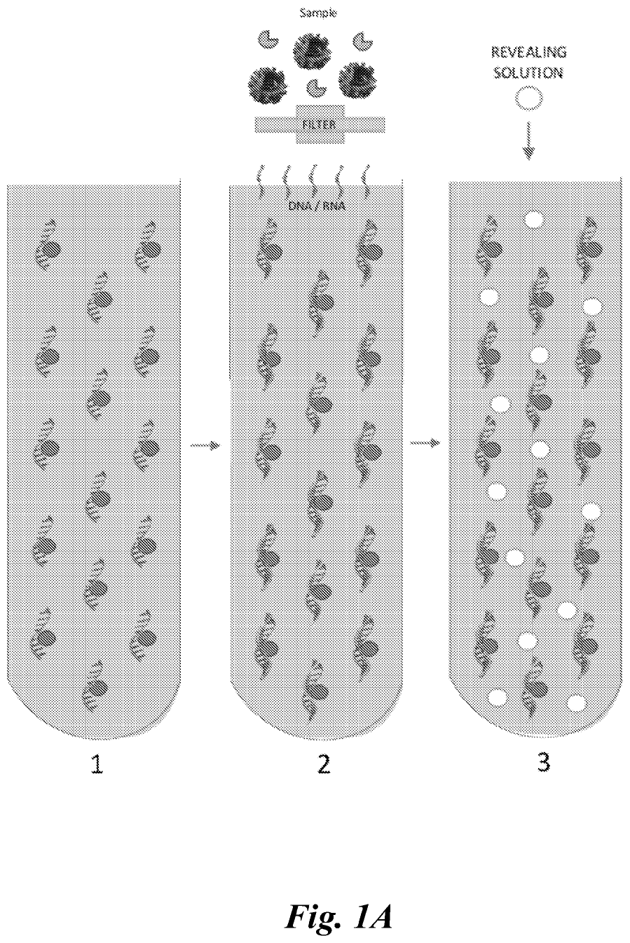

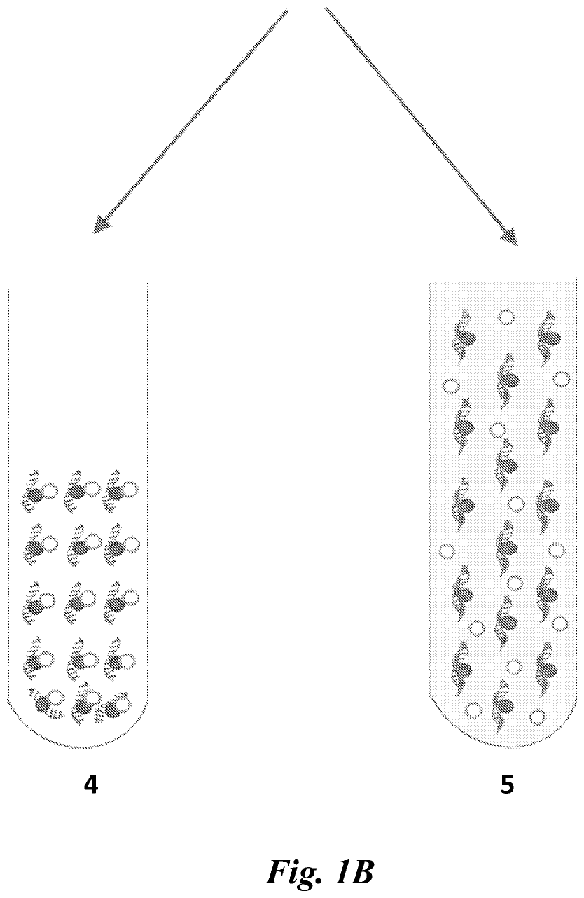

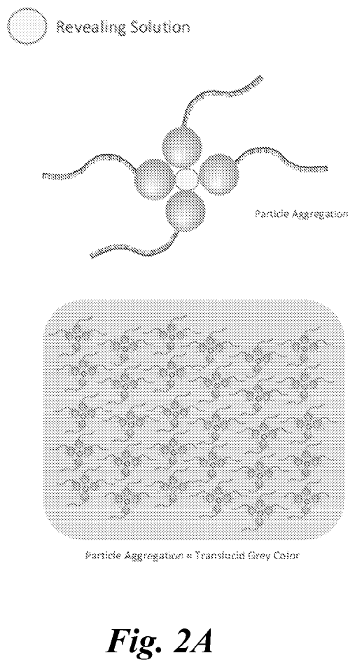

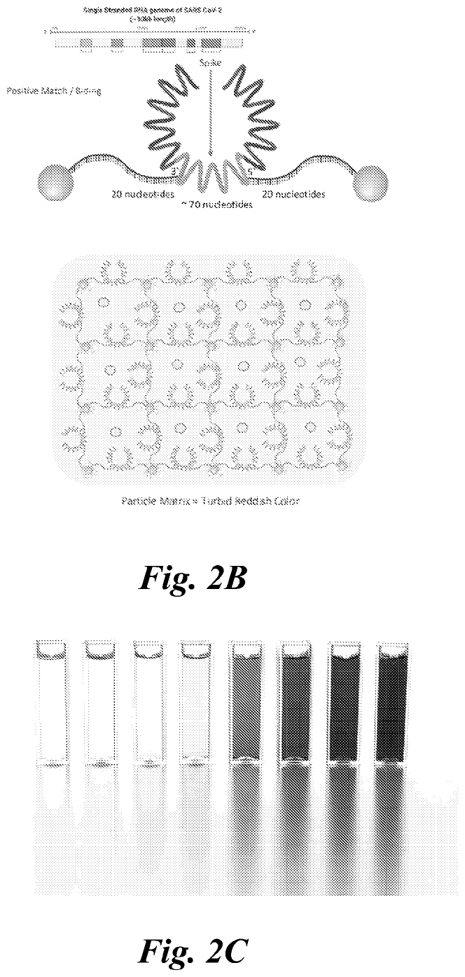

Two types of sample are analyzed via RT-PCR and use the AuNPs as a detection agent. The AuNPs comprising oligonucleotides are incubated with two different solutions, one which contains the target analyte (and is detectable via RT-PCR), and another solution that does not contain the target nucleic acid. After incubation of the AuNP with the sample solution, a revealing solution is added comprising magnesium chloride. Additionally a standard solution is generated for use as a baseline for the color measurements. FIGS. 1A-1B shows an example schematic of the process. In FIG. 1A, tube schematic 1 shows a sample tube comprising a solution of AuNPs (circle) and oligonucleotides (helix). Tube schematic 2 shows DNA/RNA from a sample is added to tube schematic 1, where the DNA/RNA is obtained by digestion or other extraction of the nucleic acids from a virus or other DNA/RNA comprising biological object. The DNA/RNA is allowed to interact with the oligonucleotides and potentially anneal. In tube schematic 3, a revealing solution is added to the tube of tube schematic 2 to induce an observable change in the tube, such as promoting condensation of the nucleic acid structures, depending on the presence of a target nucleic acid. Tube schematic 4 in FIG. 1B shows the results from tube 3, where there was no target nucleic acid present. The tube becomes clear/transparent indicating a negative result. Alternatively, tube schematic 5 shows the results from tube 3, where there is target nucleic acid present. The tube becomes opaque indicating a positive result. FIGS. 2A-2B shows an example structural schematic of the positive versus negative samples. In the negative sample, the revealing solution allows the particles to aggregate and causes the solution to turn to a translucid greyish color (FIG. 2A). Alternatively, when the oligonucleotides are able to bind a target nucleic acid, the revealing solution induces the nucleic acids and nanoparticles to forms a particle matrix, which is a turbid reddish color. FIG. 2C shows a series of cuvettes with solutions, with those of the left demonstrating the particle aggregation and a negative signal, and those on the right demonstrating a positive signal indicated by the reddish color of the solution.

Initially, the standard is defined through the identification of the primary colors of red, green and blue, respectively. From this, CIELAB color space is determined, which is a system that evaluates the elements of luminosity or clarity, tonality or hue and saturation or chromaticity. Luminosity (L*) is approximately the value of luminance (Y axis) varying from white to black, assuming the value 0 (zero) for absolute black and 100 for total white. The tonality is expressed by the primary colors, identified by the value of a*, ranging from green to red and the value of b* can vary from blue to yellow, representing in an analogous way to the perception of colors. The L*a*b* color space was created using opposite color theory, where two colors cannot be green and red at the same time, or yellow and blue at the same time. L* indicates brightness and a* and b* are chromatic coordinates. L*=Brightness; a*=red/green coordinate (+a indicates red and −a indicates green); b*=yellow/blue coordinate (+b indicates yellow and −b indicates blue); Color differences are defined by numerical comparison between the sample and the standard. The absolute differences in color coordinates between the sample and the target and are known as Deltas (Δ). Deltas for L* (ΔL), a* (Δa) and b* (Δb) can be negative (−) or positive (+). The total difference, Delta E (ΔE), however, is always positive. They are expressed as: ΔL*=difference in lighter and darker (+=lighter, −=darker); Δa*=difference in red and green (+=more red, −=more green); Δb*=difference in yellow and blue (+=more yellow, −=more blue); ΔE*=total color difference;
To determine the total color difference between the three coordinates, the following formula is used: ΔE*=[(ΔL*)2+(Δa*)2+(Δb*)2]½. Comparing the RT-PCR positive sample and the RT-PCR negative sample, each delta coordinate is significantly different and demonstrates that the color analysis is able to distinguish between a positive and negative sample.

### Example 3: Detection in Nucleic Acids in Patient Samples

A sample from a patient is collected by a sterile nasopharyngeal swab and placed immediately in a tube containing PBS medium. The sample is homogenized to disaggregate the cells from the swab. 500 μL from the collection tube is transferred to a tube containing the same lysis solution volume (v/v); The sample is homogenized and then let sit for 5 minutes for the extraction reaction to occur. The entire volume from the microtube is then removed into a syringe and a 0.22 μm filter to the syringe, and apply directly to the biosensor by dripping; 40 μL of the revealing solution is then added. After waiting for 1 minute the test is then read. Three potential results are possible. If the target nucleic acid is present, the revealed color will be dark pink/red. If the target reagent is absent, the revealed color will be blue/light gray. When it does not reveal any of the colors mentioned above, or if the color does not match one on the printed control color chart, the test is considered invalid. This result may have occurred due to insufficient amount of sample or incorrect execution of the procedure, and the test may be repeated to obtain a valid result. Negative Control Swab and Positive Control Swab may also be obtained and similar steps may be performed as quality control. FIG. 5 shows an example system for detection as used in this example. As shown, a sample is added into the entrance and allowed to mix and homogenize for 5 min before moving to a hear chamber and through a filter. The biosensor solution comprising the nanoparticles assembled to oligonucleotides is then added via a port and allowed to mix with the sample. Following mixing with the biosensor, revealing solution is then added in a port and allowed to combine with the sample (and biosensor). This resulting solution is then pushed into a new reservoir where a RGB reading (or reading of another optical parameter) can be performed. This system can use a pump (such as a syringe pump) to move the solutions through the system.

### Example 4: Generation of Nanoparticles and Detection of HPV

Nanoparticles were generated based on the protocol described in Example 1. Multiple types of nanoparticles were produced comprising nanoparticles with different ASOs. The protocol is first performed using ASO number 1 (SEQ ID NO: 1) to generate nanoparticles with ASO number 1. The process is then repeated for ASOs numbers 3, 4, and 6, in 4 (SEQ ID NO: 3, 4, and 6 respectively) vials with 2 mL of suspension, each vial with one of the ASOs. The four suspensions were mixed in equal amounts, 2 mL each, totaling 8 mL containing the mixture of particles conjugated with 4 ASOs to generate biosensor solution. The biosensor was stored in a refrigerator at 4-8° C. until use with the clinical sample.

The biosensor solution has a limpid solution appearance, is reddish, and has a maximum UV-vis absorption peak of 530 nm. Additionally, a control sequence (“PROBE”) corresponding to an HPV virus sequence was used to verify the hybridization of the ASOs to a complementary sequence. After homogenization, the solution was applied to an agarose gel electrophoresis and tested with free samples, ASOs, and PROBE, without AuNPs. The PCR used for signal amplification and differentiation of ASOs performed at two concentrations to confirm binding. The tested ASOs have 20 bases, and the PROBE has 100 bases.

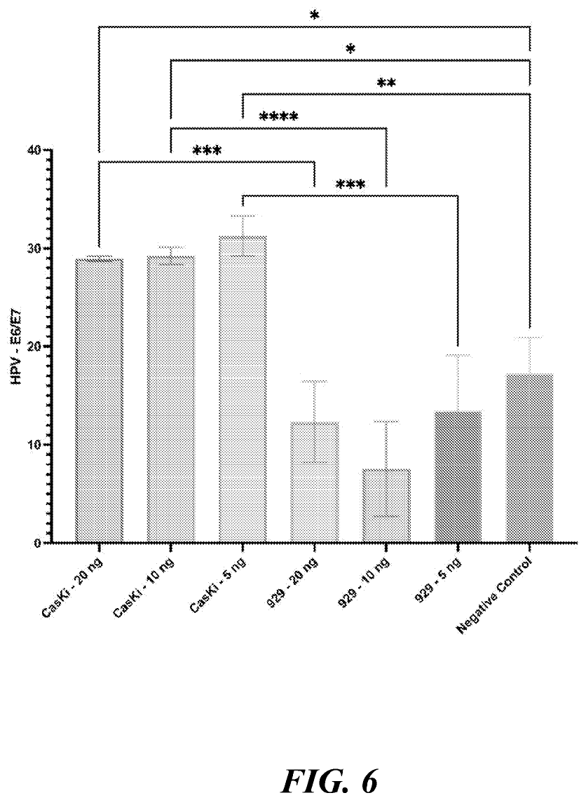

FIG. 6 shows data obtained by using the biosensor solution to detect the presence of HPV RNA. HPV RNA was expressed in the genome of CasKi (squamous cell carcinoma) cells and the biosensor solution (i.e., a solution comprising in the AuNP-ASOs) was applied to the cells. L929 cells (connective mouse tissue) were also grown which did not have HPV-RNA. RNA from the CasKi and L929 cells were extracted and assayed using the biosensor. A sample of PBS was also assayed as a negative control/background signal. The spectral data of the samples were read, parameterized, and normalized using an algorithm to generate a spectral score. As demonstrated in FIG. 6, the samples comprising extracted CasKi RNA (“CasKi −20 ng,” “CasKi −10 ng,” “CasKi −5 ng”) showed significantly higher spectral score compared to samples comprising extracted L929 RNA (“929 −20 ng,” “929 −10 ng,” “929 −5 ng”) which showed spectra similar to the sample with PBS only (“Negative control”). As such, the samples positive for the HPV were distinguishable from samples negative for HPV RNA. Additionally, assays on nucleic acids different from the target nucleic acid generated results consistent with negative samples (e.g., PBS only), demonstrating specificity in recognition of the target sequence.

### Example 5. Detection of Nucleic Acids Using Optical Density

Samples of bacteria were first generated by growing cultures for ˜16 hrs (e.g., overnight) at 37 C in BOD (Biochemical Oxygen Demand) chamber until an optical density of 0.5. The samples were initially diluted between 100× and 100,000× using 0.1% peptone water, and then made into a serial dilution curve. The diluted samples were then incubated in a dry heat bath at 95 C for 5 minutes. After incubations, the samples were placed on ice to stop the boiling. The samples were then vortexed for 10 seconds and then placed on ice. The samples were then added to a 96 well plate by pipetting 100 μl of the samples. Along with the test Samples, negative controls and biosensor only samples were also generated, based on the following table:

TABLE 2
Sample preparation guide

| | | | | Salt (e.g., |
|---|---|---|---|---|
| | | H₂O | | revealing |
| Samples | Lysate | peptone | Biosensor | solution) |
| Test sample | 100 μl | — | 140 μl | 10 μl |
| Negative Control | — | 100 μl | 140 μl | 10 μl |
| Biosensor | — | 100 μl | 140 μl | — |

The various samples were added to the plate and incubated at room temperature for 5 minutes. A revealing solution is generated by mixing 0.79 g MgCl₂ in water to generate a 50 mL total volume. 10 μl of revealing solution is added to the wells and is incubated for 2-4 minutes at room temperature and then read using an absorbance reading at 520 nm. The optical density at 520 nm is then obtained and can be analyzed using the following formula

$${2}^{ΔΔ O . D .} = \frac{{2}^{Δ ( biosensor control - sample )}}{{2}^{Δ ( biosensor control - negative control )}}$$

After the calculation was performed, a cut-off is determined (2 standard deviation below the negative control). Reading above the cut-off were considered as negative and those below the cutoff were considered positive.

### Example 6: Detection of HPV Using Optical Density Measurements

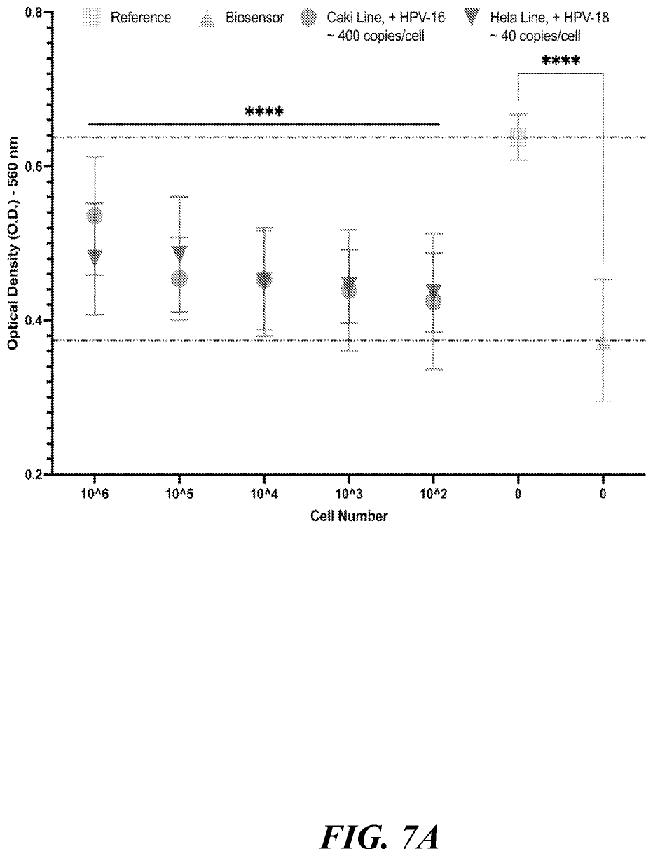

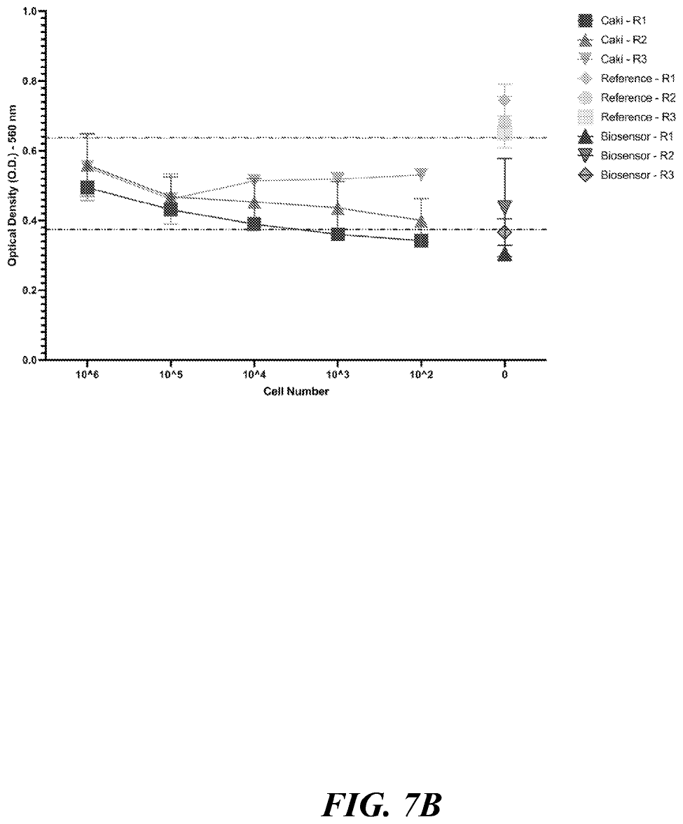

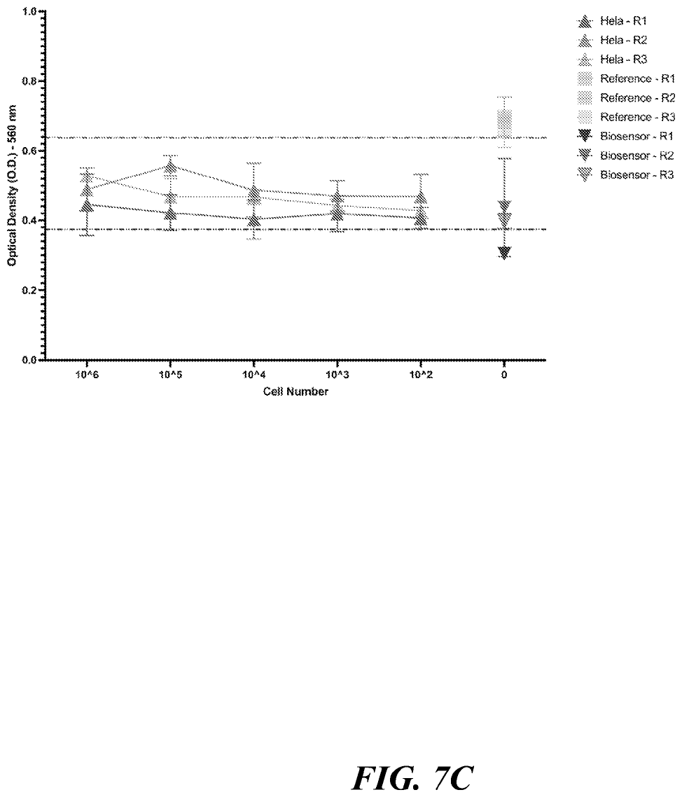

Samples of HPV were generated similarly to as described in Example 6. Using three biological replicates, samples of Caski cells lines +HPV-16 and HeLa cell lines with HPV-18 were diluted to form a serial dilution curve. The AuNP-ASO biosensors were then added to the solution and used to detect the presence of HPV at a level that was statistically significant over the negative control of the biosensor alone (i.e., without target nucleic acids present). A reference sample was also used to evaluating the colloidal dispersion and determining the range of indeterminates. FIG. 7A shows results an assay for detection of HPV using an Optical Density at 560 nm (OD₅₆₀) to detect the signal. FIG. 7B shows the individual values of the OD₅₆₀ of the biological replicates of the CasKi cells, references sensor and biosensor only samples, and FIG. 7C shows the individual values of the OD₅₆₀ of the biological replicates of the HeLa cells, references sensor and biosensor only samples. Table 3 shows a analytical performance of this assay.

TABLE 3
Analytical performance of HPV assay

| Sensitivity (rate of true positives) = (a/(a + c)) | 95.56 | |
|---|---|---|
| IC (95%) | 87.09 | 104.03 |
| Specificity (rate of true negatives) = (d/(b + d)) | 100.00 | |
| IC (95%) | 95.62 | 104.38 |
| Pravalence (real) = (a + c/(a + b + c + d)) | 81.82 | |
| IC (95%) | 76.90 | 83.46 |
| Estimated Pravalence (Test) = (a + b/(a + b + c + d)) | 78.18 | |
| IC (95%) | 72.20 | 83.46 |
| Correct Classification (Accuracy) = | 96.36 | |
| (a + d/(a + b + c + d)) | | |
| IC (95%) | 91.09 | 101.64 |
| Incorrect Classification = (b + c/(a + b + c + d)) | 3.64 | |
| IC (95%) | −1.64 | 8.91 |
| Positive predictive value (PPV) = (a/(a + b)) | 100.00 | |
| IC (95%) | 90.91 | 109.09 |
| Negative predictive value (VPN) = (d/(c + d)) | 83.33 | |
| IC (95%) | 80.13 | 86.53 |
| Kappa coefficient | K= | 0.89 |

| | |
|---|---|
| Kappa Agreement Scale | Almost Perfect |

a = number of true positives,
b = number of false positives,
c = number of false negatives,
d = number of true negatives;
IC = Confidence Interval

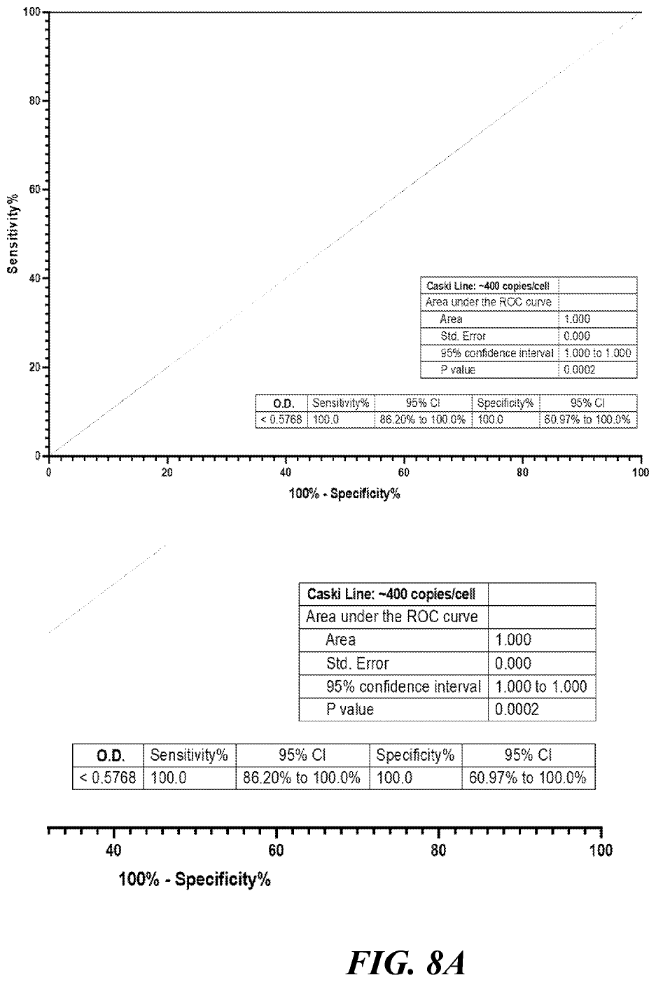

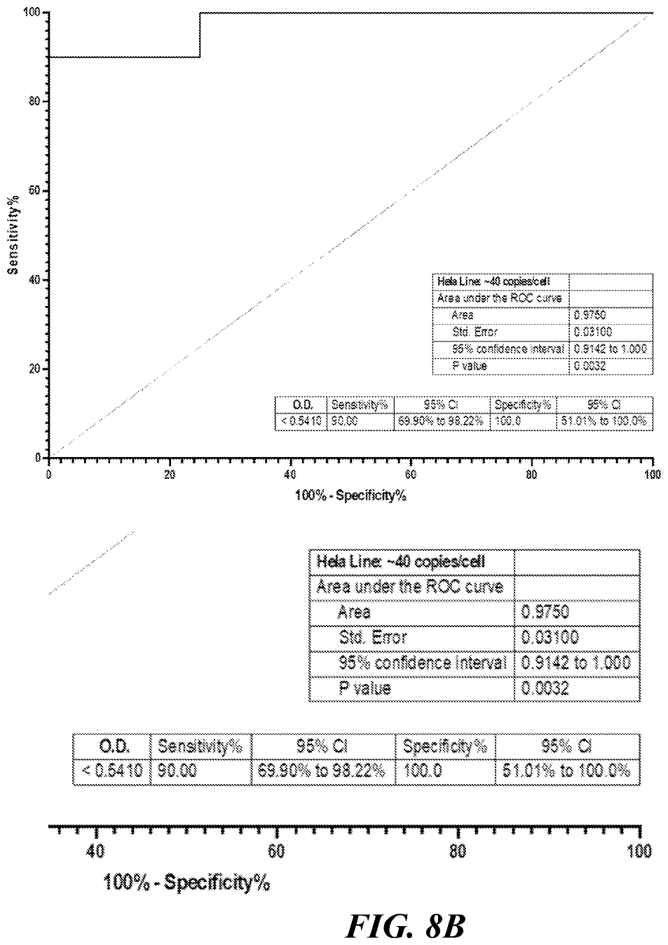

FIGS. 8A and 8B shows ROC (Receiver Operating Characteristic) curves for the dilutions of the Caski cell lines with HPV and HeLa cell lines with HPV, with an AUC (area under the curve) of 1 for the assay using the CasKi cell lines at (˜400 copies of HPV/cell) and an AUC of 0.9750 for the HeLa cell lines (˜40 copies of HPV/cell).

### Example 7. Detection of Salmonella

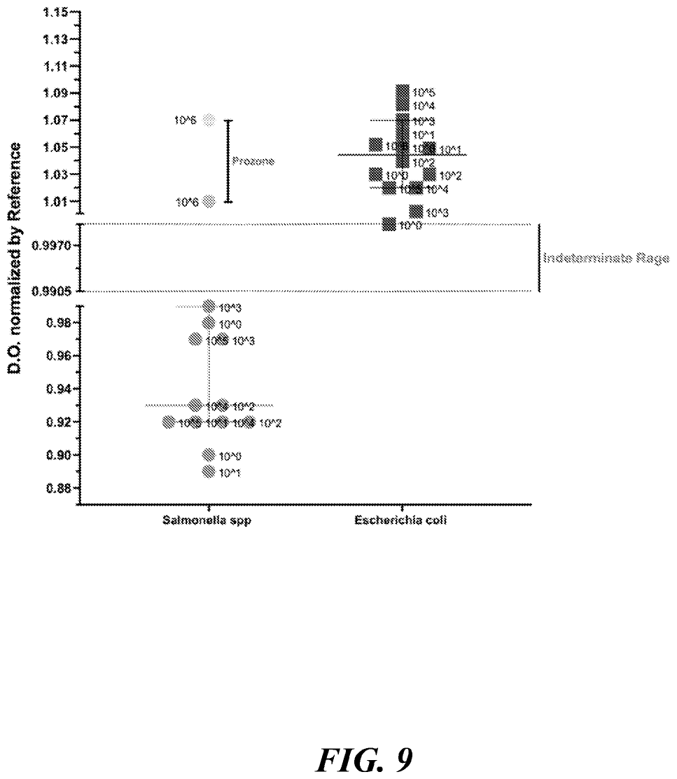

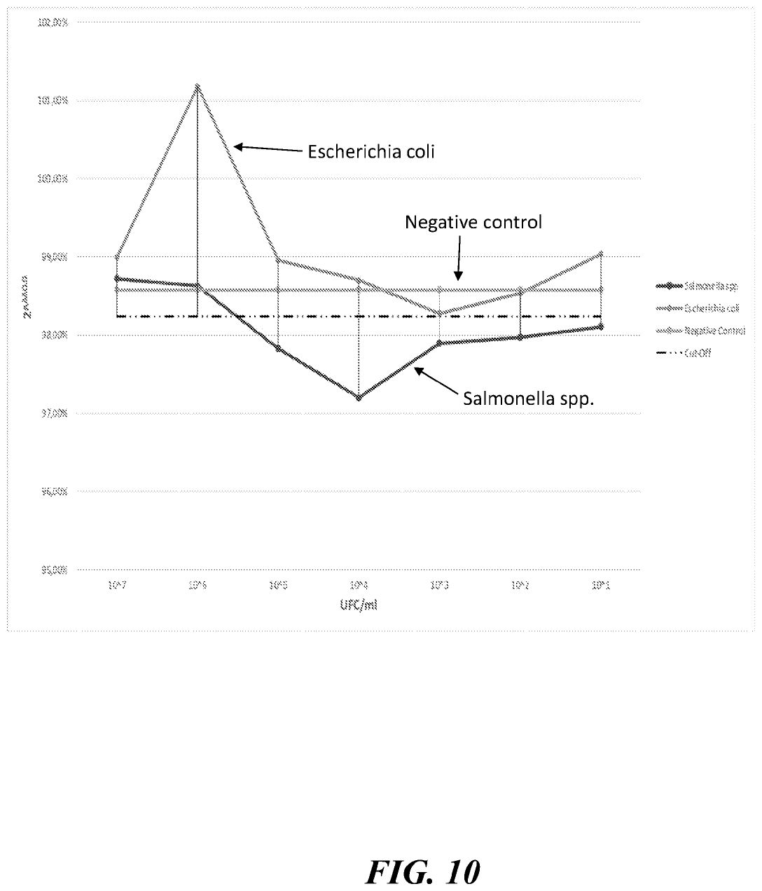

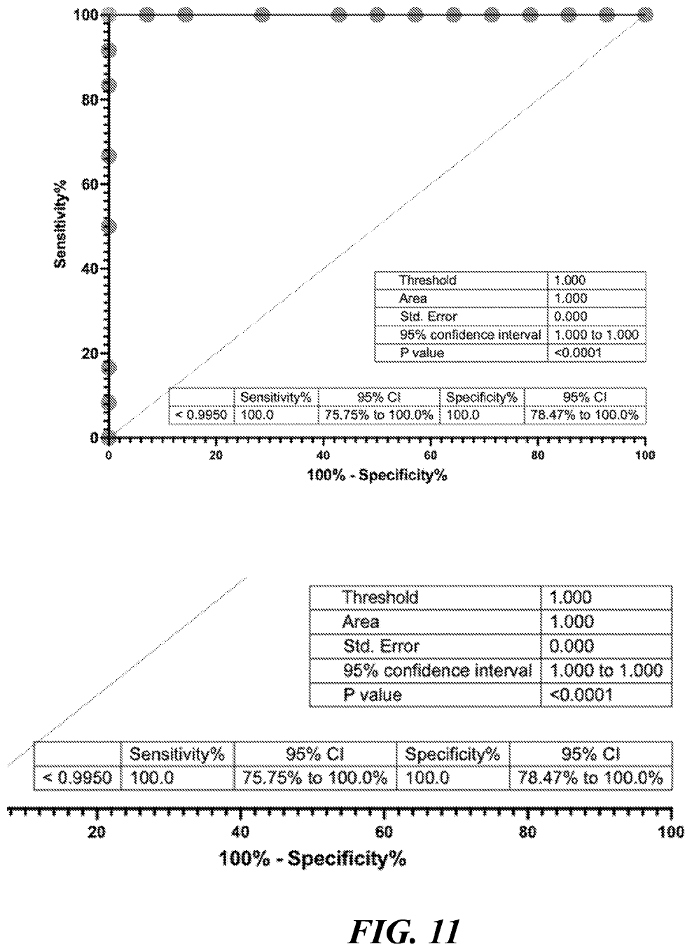

Nanoparticles were generated based on the protocol described in Example 1. Additionally, reference samples were generated. The dilutions were mixed with the AuNP-ASOs and the optical density of the samples were read. FIG. 9 shows the optical density normalized against the reference of various dilutions of Salmonella and E.coli. Of note the E.coli samples (squares) shows a normalized optical density greater than 1 whereas the Salmonella samples (circles) show a normalized optical density less than 0.9905. The Salmonella samples of 10⁶ or larger experience a prozone phenomenon and therefore generate a different signal, and signals. FIG. 10 shows a graph of the colony forming units per milliliter (“UFC/ml”) graphed against a processed optical density parameter. As shown, Salmonella samples of dilutions below 10⁶ cfu/ml were lower than the cutoff (cutoff=2 times the standard deviation of the negative control), whereas sample of E.coli were above the cut-off. FIG. 11 shows the ROC curve analysis, with an AUC of 1. Table 4 shows a analytical performance of assay for Salmonella.

TABLE 4
Analytical performance of Salmonella assay

| Sensitivity (rate of true positives) = (a/(a + c)) | 91.67 | |
|---|---|---|
| IC (95%) | 87.67 | 95.67 |
| Specificity (rate of true negatives) = (d/(b + d)) | 100.00 | |
| IC (95%) | 95.20 | 104.80 |
| Pravalence (real) = (a + c/(a + b + c + d)) | 50.00 | |
| IC (95%) | 47.45 | 52.55 |
| Estimated Pravalence (Test) = (a + b/(a + b + c + d)) | 45.83 | |
| | 43.28 | 48.38 |
| Correct Classification (Accuracy) = | 95.83 | |
| (a + d/(a + b + c + d)) | | |
| IC (95%) | 93.28 | 98.38 |
| Incorrect Classification = (b + c/(a + b + c + d)) | 4.17 | |
| IC (95%) | 1.62 | 6.72 |
| Positive predictive value (PPV) = (a/(a + b)) | 100.00 | |
| IC (95%) | 95.40 | 104.60 |
| Negative predictive value (VPN) = (d/(c + d)) | 92.31 | |
| IC (95%) | 88.08 | 96.54 |
| Kappa coefficient | K= | 0.92 |

| | |
|---|---|
| Kappa Agreement Scale | Almost Perfect |

a = number of true positives,
b = number of false positives,
c = number of false negatives,
d = number of true negatives;
IC = Confidence Interval

The method may be performed on a food sample, where the presence of Salmonella is detected in the food sample. The method may also determine the quantity of Salmonella in the food sample.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

What is claimed is:

1. A method for processing or analyzing a sample, the method comprising:
a. contacting the sample with a composition that comprises one or more nanoparticles assembled with one or more oligonucleotides to provide a test composition, wherein the one or more oligonucleotides hybridize to one or more target nucleic acids, if present, in the sample;
b. if the one or more target nucleic acids are present, forming a nanoparticle matrix comprising the one or more nanoparticles hybridized to the one or more target nucleic acids;
c. determining an optical parameter of the test composition that is indicative of the presence or absence of the one or more target nucleic acids in the sample.

2. The method of claim 1, wherein the optical parameter is determined by a color spacing analysis.

3. The method of claims 1 or claim 2, wherein the optical parameter comprises absorbance, transmission, scattering, or reflection of a light at a wavelength or a range of wavelengths.

4. The method of any one of claims 1-3, wherein the optical parameter comprises a luminosity parameter (e.g., brightness of a color), a saturation parameter (e.g., intensity of a color), or a tonality parameter (e.g., shade of a color).

5. The method of any one of claims 1-4, wherein (c) further comprises comparing the optical parameter of the test composition with a corresponding optical parameter determined from a corresponding reference composition.

6. The method of any one of claims 1-5, wherein the sample is selected from: a blood sample, a serum sample, a plasma sample, a saliva sample, a stool sample, a sputum sample, a urine sample, a semen sample, a transvaginal fluid sample, a cerebrospinal fluid sample, a sweat sample, a cell sample, a tissue sample, or a food sample.

7. The method of any one of claims 1-6, wherein (b) comprises contacting the test composition with a nanoparticle condensation agent and/or salt.

8. The method of claim 7, wherein the condensation agent comprises magnesium chloride.

9. The method of any one of claims 1-8, wherein the one or more oligonucleotides have a melting temperature (Tm) of at least about 65 degree Celsius (° C.).

10. The method of any one of claims 1-9, wherein the one or more oligonucleotides have a Tm of about 65° C. to about 75° C.

11. The method of any one of claims 1-10, wherein a nanoparticle of the one or more nanoparticles is assembled with one, two, three, four, five, or six oligonucleotide(s), optionally wherein each of the oligonucleotide(s) are the same or different.

12. The method of any one of claims 1-11, wherein the one or more nanoparticles comprise gold.

13. The method of any one of claims 1-12, wherein the one or more nanoparticles have an average size of about 10 nanometers (nm) to about 200 nm.

14. The method of any one of claims 1-13, wherein each of the one or more oligonucleotides are about 16 to about 24 nucleotides long.

15. The method of any one of claims 1-14, wherein the one or more oligonucleotides comprises two oligonucleotides, wherein the first oligonucleotide hybridizes to a first region of the target nucleic acid, and the second oligonucleotide hybridizes to a second region of the target nucleic acid.

16. The method of claim 15, wherein the distance between the first region and the second region of the target nucleic acid is about 50 to about 70 nucleotides.

17. The method of any one of claims 1-16, wherein each of the one or more oligonucleotides hybridize to about 10 to about 30 nucleotides of the one or more target nucleic acids.

18. The method of any one of claims 1-17, wherein, in the absence of one or more target nucleic acids, the one or more nanoparticles form an aggregate.

19. The method of any one of claims 1-18, wherein the one or more target nucleic acids comprise viral and/or bacterial nucleic acids.

20. The method of claim 19, wherein the one or more target nucleic acids are not coronavirus nucleic acids.

21. The method of any one of claims 19-20, wherein the one or more target nucleic acids comprises viral nucleic acids, and wherein the viral nucleic acids comprise influenza and/or a human papilloma virus nucleic acids.

22. The method of any one of claims 19-21, wherein the one or more target nucleic acids comprises bacterial nucleic acids, wherein the bacterial nucleic acids comprises Salmonella nucleic acids.

23. The method of claim 22, wherein the Salmonella comprises one or more Salmonella strains or serovars.

24. The method of claim 23, wherein the one or more Salmonella strains or serovars comprises Salmonella typhimurium, Salmonella enteritidis, Salmonella gallinarum or Salmonella pullorum, or a combination of two or more thereof.

25. The method of any one of claims 1-24, wherein the presence of the one or more target nucleic acids is associated with one or more diseases or conditions in a subject comprising the one or more target nucleic acids.

26. The method of any one of claims 1-25, wherein the one or more target nucleic acids are human papillomavirus (HPV) nucleic acids, or nucleic acids of one or more variants of HPV.

27. The method of any one of claims 1-26, wherein the one or more target nucleic acids comprise L1 capsid protein of HPV, L2 capsid protein of HPV, E6 protein of HPV, or E7 protein of HPV, or fragments and/or combinations of two or more thereof.

28. The method of any one of claims 1-27, wherein the one or more oligonucleotides comprise a sequence selected from SEQ ID NOs: 1-18, or a sequence at least 90% identical to a sequence selected from SEQ ID NOs: 1-18.

29. A composition for detecting one or more target nucleic acids, the composition comprising:
a. one or more nanoparticles assembled with one or more oligonucleotides, wherein the one or more oligonucleotides are complementary to the one or more target nucleic acids,
b. wherein, in the presence of the one or more target nucleic acids, the one or more nanoparticles form a nanoparticle matrix comprising the one or more nanoparticles and the one or more target nucleic acids, wherein the nanoparticle matrix comprises a different optical parameter compared to a solution comprising corresponding nanoparticles that are not bound to the one or more target nucleic acids.

30. A composition for detecting a target nucleic acid, the composition comprising:
a. one or more nanoparticles assembled with one or more oligonucleotides, wherein a first oligonucleotide of the one or more oligonucleotides is complementary to said target nucleic acid at a first sequence of the target nucleic acid, and wherein a second oligonucleotide of the one or more oligonucleotides is complementary to said target nucleic acid at a second sequence of the target nucleic acid, wherein the one or more nanoparticles comprise gold,
b. wherein, in the presence of the one or more target nucleic acid, the one or more nanoparticles form a nanoparticle matrix.

31. The composition of any one of claims 29-30, wherein a nanoparticle of the one or more nanoparticles is assembled with one, two, three, four, five, or six oligonucleotide(s).

32. The composition of any one of claims 29-31, wherein the one or more nanoparticles are each (e.g., independently) assembled with one, two, three, four, five, or six oligonucleotide(s).

33. The composition of any one of claims 29-32, wherein the one or more nanoparticles comprise gold.

34. The composition of any one of claims 29-33, wherein the one or more nanoparticles have an average size of about 10 nanometers (nm) to about 200 nm.

35. The composition of any one of claims 29-34, wherein the one or more oligonucleotides are about 16 to about 24 nucleotides long.

36. The composition of any one of claims 29-35, wherein the one or more target nucleic acids comprises viral nucleic acids and/or bacterial nucleic acids.

37. The composition of claim 36, wherein the one or more target nucleic acids are not coronavirus nucleic acids.

38. The composition of any one of claims 36-37, wherein the one or more target nucleic acids comprises the viral nucleic acids, and wherein the viral nucleic acids comprise an influenza virus nucleic acid and/or a human papilloma virus nucleic acid.

39. The composition of claim 36, wherein the one or more target nucleic acids comprises the bacterial nucleic acids, and wherein the bacterial nucleic acids comprise a Salmonella nucleic acid.

40. The composition of any one of claims 29-39, wherein the one or more oligonucleotides comprise a sequence selected from SEQ ID NOS: 1-18, or a sequence at least 90% identical to a sequence selected from SEQ ID NOS: 1-18.

41. A kit for identifying the presence of a target nucleic acid, the kit comprising: (i) one or more gold nanoparticles assembled with one or more oligonucleotides, (ii) a condensation solution, (iii) instructions for using said one or more gold nanoparticles assembled with one or more oligonucleotides.

42. A kit for identifying the presence of a target nucleic acid, the kit comprising: (i) the composition of any of claims 29-40, (ii) a condensation solution, (iii) instructions for using said one or more gold nanoparticles assembled with one or more oligonucleotides.
